(19) United States
(12) Patent Application Publication
(10) Pub. No.: US 2003/0028927 A1
(43) Pub. Date: Feb. 6, 2003

(54) Methods and compositions for revealing hidden genetic variation in plants

(76) Inventors: Susan Lindquist, Chicago, IL (US); Christine Queitsch, North Cambridge, MA (US); Todd A. Sangster, Chicago, IL (US)

Correspondence Address: FULBRIGHT & JAWORSKI, LLP, 1301 MCKINNEY, SUITE 5100, HOUSTON, TX, 77010-3095, US

(21) Appl. No.: 10/170,656

(22) Filed: Jun. 13, 2002

Related U.S. Application Data
(60) Provisional application No. 60/298,211, filed on Jun. 13, 2001.
(60) Provisional application No. 60/379,484, filed on May 10, 2002.

(51) Int. Cl. A01H 1/00; C12Q 1/68

(52) U.S. Cl. 800/289; 435/6

(57) ABSTRACT

The present invention regards a method of unmasking or revealing genetic variation in eukaryotic organisms, such as plants, to eukaryotic organisms, particularly plants, produced by the method, and eukaryotic organisms, particularly plants, that exhibit a phenotype (phenotype trait) masked by Hsp90 function (activity). Specifically, the present invention is directed to the detecting genetic variation in a plant by interfering with the Hsp90 buffer system. More specifically, endogenous Hsp90 activity is inhibited by drugs or genetic manipulation that results in the manifestation of pre-existing yet otherwise undetected genetic variations, such as polymorphisms.

The present invention claims priority to U.S. Provisional Patent Application Serial No. 60/298,211 filed Jun. 13, 2001 and to U.S. Provisional Patent Application Serial No. 60/379,484, filed May 10, 2002, both incorporated by reference herein in their entirety.

The government owns rights in the present invention pursuant to a grant from the National Institutes of Health, grant number NIH GM25374.

### FIELD OF THE INVENTION

The present invention is directed to the fields of plant biology and genetics. In particular, the present invention concerns identifying genetic variation in a plant. More particularly, the present invention is directed to uncovering masked genetic variation in a plant by inhibiting activity of Hsp90.

### BACKGROUND OF THE INVENTION

Evolution relies on genetic differences between individuals to remodel shapes and functions. A species, however, must also be robust to individual genetic differences requiring some kind of buffering system in the face of genetic and environmental variation to ensure normal development (canalization; Waddington 1942; Mather, 1953). Several studies from the first half of last century illustrate that changes in environmental conditions can reveal genetic variants upon which selection can act to produce heritable changes in plants and animals (assimilation; Durrant, 1962; McLaren, 1999; Waddington, 1952), presuming that under these conditions the proposed buffer system was compromised. Recent work using genetic analysis of Drosophila melanogaster provided evidence that the chaperone Hsp90 can serve as such a buffer. Remarkably, it can do so in a multitude of different morphological pathways.

Hsp90 is among the most abundant cellular proteins and essential in all eukaryotes tested. Under physiological conditions, Hsp90 binds with great specificity to a diverse highly select set of inherently unstable proteins (e.g. kinases and transcription factors). This interaction keeps them poised for activation until they are stabilized by conformational changes, such as those associated with signal transduction. The requirement for Hsp90 in many key regulatory pathways renders them sensitive to a decrease in its function (Buchner, 1999; Mayer and Bukau, 1999; Young et al., 2001).

In Drosophila, reducing Hsp90 function by mutation, pharmacological inhibition, or environmental stress, produces an extraordinary variety of morphological changes affecting virtually every structure of the fly. Most notably, the particular change observed in any fly depends on the genetic background. Thus, reducing Hsp90 function reveals a myriad of preexisting hidden polymorphisms. These polymorphisms have not yet been mapped but likely occur not only in Hsp90 target proteins themselves, but also in other steps in Hsp90-regulated pathways, and in any factors that interface with them directly or indirectly. Through selective breeding multiple polymorphisms that affected specific traits in the fly could be enriched to the point where the trait was expressed even when Hsp90 function was fully restored. Thus, by virtue of its natural function in facilitating conformational changes in key regulators of growth and development, Hsp90 allows the storage and release of genetic variation in Drosophila and provides a plausible mechanism for promoting stepwise evolutionary change.

The sessile, photoautotrophic, and self-pollinating plant Arabidopsis thaliana differs profoundly in life-style from the obligatorily outcrossing fly Drosophila melanogaster. Genetic variability of an inbreeding species is reduced by the smaller effective population size through repeated colonizing and extinction events, effects of background selection and selective sweeps caused by hitchhiking. Some genetic polymorphisms are present within Arabidopsis populations and outcrossing does occur (Estimated frequency is ˜0.3%. Abott and Gomes, 1989). However, the heterozygousity per individual is extremely low. (Abott and Gomes, 1989; Kuittinen and Savolainen, 1997). In obligatory outcrossing species such as Drosophila heterozygousity is an important means of maintaining genetic variability; inbreeding species become rapidly homozygous after acquiring a mutation or undergoing an outcrossing event. Moreover, in Drosophila development is highly canalized: the same morphologies are observed in the face of considerable environmental variation. Arabidopsis (and plants in general) are highly plastic in their development with morphologies varying dramatically with even modest changes in the environment. Thus, Arabidopsis thaliana offers an opportunity to examine whether Hsp90 is important in buffering genetic variation in an evolutionary distant species with a vastly different life style than Drosophila melanogaster. In addition, it also offers the possibility to study importance of this buffer for developmental plasticity and its effects on the positive and negative correlations between heterozygousity and fitness created by mixing genomes of distinct ecotypes (through crosses) that have been evolving independently for a long period.

### SUMMARY OF THE INVENTION

There is great interest in developing new plant varieties, particularly those which exhibit characteristics of interest, such as larger leaves and flowers, longer roots, increased tolerance to adverse growing conditions and higher moisture content. Traditional breeding and selection methods and recombinant DNA technology are useful for these purposes. However, it would be advantageous to have additional methods, particularly methods that speed the identification of desirable traits or avoid public concern about transgenic (genetically modified) plants.

Genetic variability is the basis of evolutionary change. Genetic variability levels and patterns differ in inbreeding plants compared to obligatorily outcrossing organisms. Unlike animals, plants exhibit great developmental plasticity by coupling external environmental cues, such as light, tightly to developmental pathways. Existing natural variation in these pathways is of universal interest as a resource for breeding especially in the light of the current public disapproval of transgenic organisms.

This invention relates to a method of unmasking or revealing genetic variation in eukaryotic organisms, such as plants; eukaryotic organisms, particularly plants, produced by the method, and eukaryotic organisms, particularly plants, that exhibit a phenotype (phenotypic trait) masked by Hsp90 function (activity). In particular embodiments, the method of the present invention is a method of identifying or revealing genetic variation in plants by reducing Hsp90 function sufficiently (to a sufficient extent) to reveal or unmask a phenotype or phenotypes (phenotypic characteristic(s)) of a plant that is not exhibited by the plant in the absence of inhibition of Hsp90 function (is not exhibited by the plant in which Hsp90 function is at or above a threshold level below which genetic variation, normally hidden in the plant, is revealed or unmasked). In the method, Hsp90 buffer capacity is reduced to the extent that cryptic genetic variation in a plant is unmasked or revealed. In one embodiment, the present invention is a method of identifying (detecting) genetic variation in the genome of a plant, comprising inhibiting Hsp90 function in a plant cell to an extent sufficient to reveal genetic variation not detectable in the plant cell in the absence of inhibition of Hsp90 function; maintaining the plant cell under conditions appropriate for growth of the cell and production of a plant and determining whether the resulting plant exhibits a phenotype different from a control plant produced from the same cell type, in which Hsp90 function is not inhibited, wherein if the resulting plant exhibits a different phenotype from the phenotype of the control plant, genetic variation has been identified. The plant cell is, in specific embodiments, a plant seed cell. Leaf cell, root cell or a cell from another plant component. The plant seed cell is, for example, an F1, F2 or later generation seed cell.

In a further embodiment, the present invention is a method of identifying genetic variation in a plant, comprising growing a seed under conditions that inhibit Hsp90 function in the seed to an extent sufficient to reveal genetic variation in the seed not detectable in the absence of inhibition of Hsp90 function, thereby producing a plant and determining whether the plant exhibits a phenotype different from the phenotype of a plant grown from the same type of seed in the absence of inhibition of Hsp90, wherein if the phenotypes of the two plants differ from one another, genetic variation has been identified.

The conditions that inhibit Hsp90 function or activity (inhibit Hsp90 buffering capacity) include any that affect protein folding. In specific embodiments, the condition is pharmacologic (use of a drug or chemical that inhibits Hsp90). In further specific embodiments, the drug used is geldanamycin or radicicol. Alternatively, environmental conditions (such as, but not limited to, temperature, moisture, light intensity and/or duration, carbon dioxide levels, salt concentrations) can be altered to inhibit Hsp90 function. Other growth conditions (such as substrate density) can also be altered to inhibit Hsp90 function. In a specific embodiment, Hsp90 function is inhibited by an increase in the temperature at which the method is carried out

The present method is applicable to a wide variety of eukaryotic organisms and particularly to plants, including but not limited to, corn, cotton, rice, barley, oats, canola, soybean, wheat, rye, tobacco, sorghum, Arabidopsis thaliana, sunflower, alfalfa,. tomato, potato, sugar beef cassaya, broccoli, cauliflower, peanut, olive tree, grass, rose, carnation, daisy, orchid, tulip, iris, palm, fern, focus, evergreen, ivy, grape, hops, aloe vera, opium, poppy, sweet potato, yam, Echinacea, witch hazel and gingko biloba.

A further embodiment of the present invention is plants produced by the method of the present invention. Such plants exhibit phenotypic characteristics (a phenotype(s)) not exhibited by the corresponding (control) plant in which Hsp90 function is not inhibited to the extent sufficient to unmask or reveal cryptic genetic variation.

Plants of the present invention can be crossed (with plants of the same type or with plants of a different type/outcrossed) to enrich for a phenotype(s) identified or revealed by the method of the present invention. Such enriched plants, also the subject of the invention, exhibit a characteristic(s) not exhibited or exhibited to a lesser extent than in the corresponding plant in which Hsp90 function has not been reduced (control plants).

A wide variety of phenotypic characteristics or traits can be identified or revealed by the present method. These include, but are not limited to, leaf size and shape, flower size and shape, root length, sugar or moisture content of fruit, tolerance to salt concentrations, resistance to pathogens, pigmentation of plant parts, flavor and fragrance.

Thus, the present invention is based, in part, on the work of the inventors utilizing the model plant Arabidopsis thaliana to investigate buffering by the chaperone Hsp90 of natural variation in developmental pathways. Hsp90-dependent variation was found in morphological pathways and in the response to growth in the dark of CVI/Ler RI lines. Further, heterozygous advantage was observed for F1 progeny of ecotype crosses over homozygous parental ecotypes when Hsp90 buffering function was compromised.

In the environment, the buffering function of Hsp90 becomes critical for the inbreeding of plants such as Arabidopsis, which rarely contain heterozygous alleles to maintain genetic variability and therefore competence to adapt to a changing environment.

A skilled artisan recognizes, based on the teachings provided herein, that inhibition of Hsp90 activity facilitates detection of genetic variation in plants and other organisms. In particular embodiments, the inhibition of Hsp90 is through pharmacological means or by decreasing expression of the endogenous Hsp90 gene or genes, such as by knockout. A skilled artisan also recognizes that, in the embodiment wherein a knockout of Hsp90 is the cause of the inhibition of Hsp90 activity, once the trait is uncovered and stably propagated, the wildtype Hsp90 nucleic acid sequence may be integrated back into the genome to circumvent any concerns regarding genetically modified organisms.

In an embodiment of the present invention, inhibition of Hsp90 uncovers pre-existing genetic variation, such as in polymorphisms, which manifest as developmental morphology and/or morphology alterations in response to an environmental stimulus, such as temperature, light, dark, gravity, salinity, pH, touch, sound, exposure to metal, or a combination thereof. A skilled artisan recognizes that, in a specific embodiment, an environmental stimulus influences Hsp90 activity by providing an overabundance of substrates and therefore titrating away available Hsp90 molecules for activity. Inhibition of Hsp90 activity either by mutation or by drugs enhances or mimics this situation.

Thus, in an embodiment of the present invention, there is a method of detecting genetic variation in a plant genome comprising inhibiting Hsp90 activity in at least one plant cell. In a specific embodiment, the method is further defined as a method of determining a plant polymorphism. In another specific embodiment, the inhibiting of Hsp90 activity lowers a threshold for manifestation of a pre-existing genetic polymorphism in the plant cell. In a specific embodiment, the at least one plant cell is in cell culture, a seed, or a plant.

In a specific embodiment of the present invention, inhibiting Hsp90 comprises administering an Hsp90-inhibiting agent to a plant cell. In a specific embodiment, the Hsp90-inhibiting agent is administered to a plant cell that is in cell culture. In an additional specific embodiment, the method further comprises developing a plant from the plant cell. In another specific embodiment, the Hsp90-inhibiting agent is administered to a plant cell that is in a seed, and in a further specific embodiment, the method further comprises developing a plant from the seed.

In a specific embodiment of the present invention, the Hsp90-inhibiting agent is administered to a plant cell that is in a plant. In another specific embodiment, the Hsp90-inhibiting agent is administered to the plant after the plant has sprouted from a seed. In an additional specific embodiment, the Hsp90-inhibiting agent inhibits ATPase activity of Hsp90. In another specific embodiment, the Hsp90-inhibiting agent is geldanamycin or radicicol. In an additional specific embodiment, the Hsp90-inhibiting agent is photosensitive.

In another specific embodiment of the present invention, the Hsp90 activity is inhibited by inhibiting expression of a nucleic acid sequence that encodes Hsp90. In an additional specific embodiment, the method is further defined as a method of inhibiting transcription of a nucleic acid sequence that encodes Hsp90 or of inhibiting translation of Hsp90. A skilled artisan recognizes that the inhibition of transcription or translation may be through direct or indirect means. That is, the substrates of the processes may be affected to produce the inhibition, or the machinery and related entities may be affected instead of or in addition to affected substrates. In a specific embodiment, the Hsp90 activity is inhibited by decreasing Hsp90 half-life or by knocking out a gene encoding Hsp90 in the cell's genome.

In an additional specific embodiment of the present invention, the genetic variation is detected by examining the phenotype of the plant cell. In a specific embodiment, examining the phenotype of the plant cell comprises examining the morphology or physiology of the cell. In another specific embodiment, examining the phenotype of the plant cell comprises examining the morphology or physiology of a plant comprising the cell. In an additional specific embodiment, examining the phenotype of the plant cell comprises examining the morphology of a plant comprising the cell. In another specific embodiment, examining the phenotype comprises examining cotyledon morphology, adult leaf morphology, hypocotyl morphology, root morphology, root hair morphology, and/or rosette morphology. In another specific embodiment, examining the phenotype of the plant cell comprises examining the physiology of a plant comprising the cell.

In an additional specific embodiment of the present invention, examining the phenotype comprises examining anthocyanin accumulation and/or increased response to gravity. In another specific embodiment, examining the phenotype comprises exposing a plant comprising the cell to a stimulus and assaying the plant for a reaction to the stimulus, such as temperature, light, gravity, salinity, metal, touch, sound, humidity, nutrient concentration, growing conditions, dark, and/or a change in any of these.

In another specific embodiment of the present invention, the genetic variation is detected by examining the genotype of the cell. In specific embodiment, the plant cell is a cell of a monocot or of a dicot. Examples include cotton, rice, barley, oats, canola, soybean, corn, wheat, rye, tobacco, sorghum, Arabidopsis thaliana, sunflower, alfalfa, tomato, potato, sugar beet, cassaya, broccoli, cauliflower, peanut, olive tree, grass, rose, carnations, daisies, orchids, tulips, irises, palms, ferns, ficus, evergreen, ivy, grapes, hops, aloe vera, opium poppy, sweet potatoes, yams, Echinacea, witch hazel, or Gingko biloba. In a specific embodiment, the plant cell is a cell of Arabidopsis thaliana. In another specific embodiment, the plant cell is a cell of a crop plant, such as cotton, corn, sorghum, soybean, tobacco, rice, canola, wheat, rye, spinach, peanut, or mustard.

In a specific embodiment of the present invention, the method is further defined as a method of detecting genetic variation among a plurality of plant cells, a method of detecting genetic variation among a plurality of plant cells comprised in a single plant, or a method of detecting genetic variation among a plurality of plant cells comprised in a plurality of plants.

In another embodiment of the present invention, there is a method of detecting genetic variation in a genome, comprising inhibiting Hsp90 activity in at least one cell. In a specific embodiment, the method is further defined as a method of determining a polymorphism. In a specific embodiment, the inhibiting Hsp90 activity lowers a threshold for manifestation of a pre-existing genetic polymorphism in the cell. In another specific embodiment, the at least one cell is in cell culture, in embryonic tissue, in a seed, or comprised in an organism.

In an additional specific embodiment, inhibiting Hsp90 comprises administering an Hsp90-inhibiting agent to a cell. In a further specific embodiment, the Hsp90-inhibiting agent is administered to a cell that is in cell culture. In another specific embodiment, the method further comprises developing an organism from the cell. In an additional specific embodiment, the Hsp90-inhibiting agent is administered to a cell that is in an embryo. In a further specific embodiment, the method further comprises developing an organism from the embryo. In an additional specific embodiment, the Hsp90-inhibiting agent is administered to a cell that is in an organism. In another specific embodiment, the Hsp90-inhibiting agent inhibits ATPase activity of Hsp90. In further specific embodiments, the Hsp90-inhibiting agent is geldanamycin or radicicol. In another specific embodiment, the Hsp90-inhibiting agent is photosensitive. In a further specific embodiment, the Hsp90 activity is inhibited by inhibiting expression of a nucleic acid sequence that encodes Hsp90.

In another specific embodiment of the present invention, the method is further defined as a method of inhibiting transcription of a nucleic acid sequence that encodes Hsp90, of inhibiting translation of Hsp90, of inhibiting Hsp90 activity by increasing Hsp90 degradation, of inhibiting Hsp90 activity by decreasing Hsp90 half-life, or of inhibiting Hsp90 activity by knocking out a gene encoding Hsp90 in the cell's genome.

In an additional specific embodiment, genetic variation is detected by examining the phenotype of the cell, such as by examining the morphology or physiology of the cell, by examining the morphology or physiology of an organism comprising the cell, by examining the phenotype of the cell comprises examining the morphology of an organism comprising the cell, by examining the physiology of an organism comprising the cell, by examining the phenotype comprising exposing an organism comprising the cell to a stimulus and assaying the organism for a reaction to the stimulus. In a specific embodiment, the stimulus is temperature, light, gravity, salinity, metal, touch, sound, humidity, nutrient concentration, growing conditions, dark, and/or a change in any of these.

In a specific embodiment of the present invention, the genetic variation is detected by examining the genotype of the cell. In specific embodiments, the cell is a plant cell, an animal cell, a vertebrate cell, a mammalian cell, a human cell, a mouse cell, an invertebrate cell, or a fly cell. In a specific embodiment, the method is further defined as a method of detecting genetic variation among a plurality of cells. In another specific embodiment, the method is further defined as a method of detecting genetic variation among a plurality of cells comprised in a single organism. In an additional specific embodiment, the method is further defined as a method of detecting genetic variation among a plurality of cells comprised in a plurality of organisms.

In another embodiment of the present invention, there is a method of identifying genetic variation in the genome of a plant, comprising inhibiting Hsp90 function in a plant cell to an extent sufficient to reveal genetic variation not detectable in the plant cell in the absence of inhibition of Hsp90 function; maintaining the plant cell under conditions appropriate for growth of the cell and production of a plant and determining whether the resulting plant exhibits a phenotype different from a control plant produced from the same cell type, in which Hsp90 function is not inhibited, wherein if the resulting plant exhibits a different phenotype from the phenotype of the control plant, genetic variation has been identified. In a specific embodiment, the plant cell is a plant seed cell. In a further specific embodiment, the plant seed cell is an F1 seed cell. In a specific embodiment, the plant cell is a cell from a plant selected from the group consisting of: corn, cotton, rice, barley, oats, canola, soybean, wheat, rye, tobacco, sorghum, Arabidopsis thaliana, sunflower, alfalfa. tomato, potato, sugar beef cassaya, broccoli, cauliflower, peanut, olive tree, grass, rose, carnation, daisy, orchid, tulip, iris, palm, fern, focus, evergreen, ivy, grape, hops, aloe vera, opium, poppy, sweet potato, yam, Echinacea, witch hazel and gingko biloba.

In an additional embodiment of the present invention, there is a method of identifying genetic variation in a plant, comprising growing a seed under conditions that inhibit Hsp90 function in the seed to an extent sufficient to reveal genetic variation in the seed not detectable in the absence of inhibition of Hsp90 function, thereby producing a plant and determining whether the plant exhibits a phenotype different from the phenotype of a plant grown from the same type of seed in the absence of inhibition of Hsp90, wherein if the phenotypes of the two plants differ from one another, genetic variation has been identified. In a specific embodiment, the seed is a seed from a plant selected from the group consisting of: corn, cotton, rice, barley, oats, canola, soybean, wheat, rye, tobacco, sorghum, Arabidopsis thaliana, sunflower, alfalfa. tomato, potato, sugar beef cassaya, broccoli, cauliflower, peanut, olive tree, grass, rose, carnation, daisy, orchid, tulip, iris, palm, fern, ficus, evergreen, ivy, grape, hops, aloe vera, opium, poppy, sweet potato, yam, echinacea, witch hazel and gingko biloba. In another specific embodiment, the conditions that inhibit Hsp90 function are selected from the group consisting of: growth in the presence of a chemical agent that inhibits Hsp90 function and growth in the presence of an environmental change. In a further specific embodiment, the chemical agent is a drug and the environmental change is a moderate temperature increase or an increase in density of substratum on which the seed is grown. In an additional specific embodiment, the drug is geldanamycin or radicicol.

In another embodiment of the present invention, there is a method of revealing genetic variation in a plant, wherein the genetic variation is not revealed in the plant in the presence of normal Hsp90 function, comprising growing seeds from which the plant grows under conditions that cause inhibition of Hsp90 function in the seeds sufficient to produce plants that exhibit a phenotype not exhibited by plants grown from the seeds in the absence of the conditions that cause inhibition of Hsp90 function, whereby genetic variation in the plant is revealed. In a specific embodiment, the conditions that cause inhibition of Hsp90 function comprise growing the seeds in the presence of a drug that inhibits Hsp90 function. In an additional specific embodiment, the drug is selected from the group consisting of geldanamycin and radicicol. In another specific embodiment, the conditions that cause inhibition of Hsp90 function comprise growing the seeds under a temperature sufficiently elevated to inhibit Hsp90 function.

Other and further objects, features and advantages would be apparent and eventually more readily understood by reading the following specification and by any examples of the presently preferred embodiments of the invention given for the purpose of the disclosure.

### DETAILED DESCRIPTION OF THE INVENTION

It will be readily apparent to one skilled in the art that various substitutions and modifications may be made in the invention disclosed herein without departing from the scope and spirit of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, the term “plant” refers to any of various eukaryotic multicellular organisms of the kingdom Plantac. In a specific embodiment, the plant is photosynthetic and, in an alternative embodiment, includes organisms such as algae. In other specific embodiments, the plant produces an embryo, contains a chloroplast, has cellulose cell walls and/or lacks locomotion. In an alternative embodiment, a bacteria has genetic polymorphisms revealed through Hsp90 inhibition.

Hsp90 chaperones the maturation of many regulatory proteins and, in Drosophila, buffers genetic variation in morphogenetic pathways. Levels and patterns of genetic variation differ greatly between such obligatory out-breeding species as fruit flies and self-fertilizing species like the plant Arabidopsis thaliana. In further contrast, plant development is much more plastic, coupled to environmental cues. As described herein, in Arabidopsis accessions and recombinant inbred lines, reducing Hsp90 function produced an array of morphological phenotypes that were dependent upon underlying genetic variation. The strength and breadth of Hsp90's effects on the buffering and release of genetic variation indicates it has an impact on evolutionary processes, in a particular embodiment. The inventors also demonstrate that Hsp90 strongly influenced morphogenetic responses to environmental cues and buffered normal development from destabilizing effects of stochastic processes. Manipulating Hsp90's buffering capacity offers a powerful tool for harnessing cryptic genetic variation and for elucidating the interplay between genotypes, environments, and stochastic events on developmental stability.

Thus, the present invention concerns uncovering masked or hidden genetic variation in a plant by interfering with the Hsp90 buffer system. A skilled artisan recognizes multiple Hsp90 genes may be present in a plant genome and furthermore is cognizant of efficient means to inhibit them. For example, administration of a drug which interferes with an Hsp90 function, such as its ATPase function, would target all Hsp90 proteins with that particular moiety. Alternatively, the Hsp90 activity may be affected at the nucleic acid level, such as by knocking out at least one of the genes, and in a preferred embodiment all Hsp90 genes are affected. One technique to affect multiple, related genes is by cosuppression, a method well known in the art.

The methods of the present invention may in some embodiments utilize nucleic acid or amino acid sequences, which are readily obtainable by a skilled artisan. For example, the Hsp90 sequence or sequences in a plant may be genetically modified to result in partial or complete loss of functional Hsp90, such as by knocking out the endogenous gene or genes. To employ this, the Hsp90 sequences may need to be determine the Hsp90 sequence from these species, many of which may be retrieved from the publicly available National Center for Biotechnology Information's GenBank database or from commercially available sources, such as Celera Genomics, Inc. (Rockville, Md.). The following examples are provided and are followed by their GenBank Accession No. An example of an Hsp90 nucleic acid sequence is SEQ ID NO:1 (Y07613). An example of an Hsp90 amino acid sequence is SEQ ID NO:2 (CAA68885). Other examples of Hsp90 nucleic acid sequences include SEQ ID NO:3 (U55859); SEQ ID NO:4 (AF123259); SEQ ID NO:5 (AB037681); SEQ ID NO:6 (L14594); SEQ ID NO:7 (Z30243); and SEQ ID NO:8 (X67960). One skilled in the art understands that using standard molecule biology techniques one can obtain specific Hsp90 sequences from other species. In specific embodiments, the Hsp90 from one organism is utilized to genetically modify the Hsp90 from another organism.

In specific embodiments, the Hsp90 is inhibited through genetic modification of a plant, such as modification of a cell, a seed, plant tissue, a seedling, a fully-grown plant, and so forth. In further specific embodiments, the modification genetically alters the plant DNA and preferably includes any genetic modification that results in a non-functional (partially or fully), a reduced level of protein, or a substantially absent protein. In specific embodiments, the genetic modification is referred to as a knock-out, wherein part or all of at least one endogenous copy of the gene is rendered non-functional, such as through replacement with another sequence.

In specific embodiments, the plant comrising the inhibited Hsp90 is selected from the group consisting of a cereal, grass, an ornamental plant, a crop plant, a food plant, an oil-producing plant, a synthetic product-producing plant, an environmental waste absorbing plant, a plant used for alcohol, a medicinal plant, a plant used for recreational purposes, a plant used for animal feed, a biomass renewable energy source plant.

Cereal is herein defined as, a grass which has starchy grains used for food. Examples of cereals are wheat, rye, barley, rice and oats. A grass as used herein is defined as a member of the grass family or any plant with slender leaves characteristic of the grass family. Examples of grasses are St. Augustine, hybrid Bermuda grass, rye grass is commercially available through a florist, Zoysiagrass, turfgrass and coastal Bermuda grass.

An ornamental plant is herein defined as a plant used for decorative purposes, such as is commercially available through a florist. Examples of ornamental plants include flowering plants, palms, ferns, woody plants, shrubs, ficus, evergreens and ivy.

A crop plant is herein defined as a cultivated plant and/or agricultural produce, such as grain, vegetables, legumes or fruit. Examples of crop plants include cotton, corn, grapes, sorghum, soybean, tobacco, rice, canola and mustard.

A food plant is herein defined as a plant of which part is consumed. The part for consumption may be the leaves, flowers, seeds, stems, or roots. Examples of food crops include potatoes, corn, rice, peanuts and wheat.

An oil-producing plant is herein defined as a plant of which part is utilized for oil production for consumption purposes. Examples of oil-producing plants include canola, soybean, corn, peanut, olive trees and vegetables.

A synthetic-product producing plant as used herein is defined as a plant which has been engineered to produce a synthetic product such as a plastic. For example, and as taught by Poirier et al. (1995), a plant may be altered to accumulate a plastic such as polyhydroxyalkanoates (PHAs) by expressing a nucleic acid associated with its synthesis. In an alternative embodiment, the synthetic product produced by the plant is a medicament.

An environmental waste-absorbing plant as used herein is defined as a plant which has been altered to remove environmental wastes or toxins from the environment, including soil, air or water. For example, and as taught by Bizily et al. (1999) and Bizily et al. (2000), a nucleic acid sequence is inserted into a plant genome which facilitates growth in environmentally toxic conditions and removal of the waste product or toxin present. In a specific embodiment, a bacterial nucleic acid sequence is utilized to provide such a resistance. An environmentally toxic condition is herein defined as any condition in which a pollutant, waste product, toxin, or environmentally hazardous chemical or composition is present. Examples of toxic conditions include excess mercury or unacceptable levels of radioactivity.

An alcohol plant as used herein is defined as a plant of which at least part is utilized in the production of an alcoholic beverage. Examples include grapes, hops, barley, rice, corn, grain, and wheat. Examples of alcoholic beverages include beer, wine, liquor, sake and liqueurs.

A medicinal plant as used herein is defined as a plant of which at least part is utilized for consumption or manufacture of a medicament, such as a medicine, vitamin or health-improving composition. An example is aloe vera, opium poppy (Papaver somniferum), diosgenin, derived from various species of yam (Dioscorea spp.) and used to manufacture progesterone, Echinacea, witch hazel and Ginkgo biloba. In a specific embodiment, the plant has been altered to contain a vaccine or composition capable of being consumed as part of the plant and which has prophylactic or medicinal purposes. In another embodiment, the medicinal plant is used for alleviating undesirable side effects from a separate medicine or health-improving composition, or from a medical procedure. Examples of side effects include nausea and/or vomiting, hives or pain. Another example of a medicinal plant is a nutriceutical. A nutriceutical as used herein is defined as an herb or any plant used in the treatment of disease or a medical condition. Examples of nutriceuticals include chamomile, Echinacea, garlic, gingko, ginseng, kava kava, St. John's Wort, willow bark, and tumeric.

A recreational plant as used herein is defined as a plant which is utilized for leisure, recreation, past time or other similar activities. A specific embodiment, includes a grass used for a park, golf course or a sport-playing field.

An animal feed plant as used herein is defined as a plant of which at least part is utilized in the manufacture of feed for animals. Examples of such plants are sorghum, corn and soybean. The plants may be used in a mixture of ingredients for the feed. Examples of animals which may consume such feed include cows, horses, sheep, pigs and chickens.

A biomass renewable energy source plant is defined as a plant having or producing material (either raw or processed) that comprises stored solar energy that can be converted to electricity or fuel. In general terms, this includes fast-growing trees and grasses, like hybrid poplars or switchgrass; agricultural residues, like corn stover, rice straw, wheat straw, or used vegetable oils; or wood waste, such as sawdust and tree prunings, paper trash and yard clippings.

Specific examples of biomass renewable energy source plants include aleman grass; alfalfa; annual ryegrass; argan tree; babassu palm; bamboo; banana; black locust; broom; brown beetle grass; buffalo gourd; cardoon; cassaya; castor oil plant; coconut palm; common reed; cordgrass; cotton; Cuphea; eucalyptus; giant knotweed; giant reed; groundnut; hemp; jatropha; jojoba; kenaf; leucaena; lupins; meadow foxtail; miscanthus; neem tree; oil palm; olive tree; perennial ryegrass; pigeonpea; poplar; rape; reed canarygrass; rocket; root chicory; rosin weed; safflower; safou; salicornia; sheabutter tree; sorghum (Fibre); sorghum (Sweet); sorrel; soybean; sugarcane; sunflower; sweet potato; switchgrass; tall fescue; tall grasses; timothy; topinambur; water hyacinth; white foam, willow; cereals (barley, maize, oats, rye, Triticale, wheat); and pseudocereals (amaranthus, buckwheat, quinoa).

Although one skilled in the art could apply the methods and compositions as described herein to any species, specific examples include the following: cotton; rice; barley; oats; canola; soybean; corn; wheat; rye; tobacco; sorghum; Arabidopsis thaliana; clover; sunflower; alfalfa; tomato; potato; sugar beet; cassaya; broccoli; cauliflower; spinach, peanut; olive tree; grass, such as St. Augustine, hybrid Bermuda grass, rye grass, Zoysiagrass, turfgrass, and coastal Bermuda grass; flowering plants, such as roses carnations, daisies, orchids, tulips, and irises; palms; ferns; woody plants, shrubs, ficus, evergreen, ivy; grapes; hops; aloe vera; opium poppy; sweet potatoes; yams; Echinacea; witch hazel; and Gingko biloba; trees; ornamentals; vegetable-bearing plants; and fruit-bearing plants.

I. Hsp90-Inhibiting Agent

In specific embodiments of the present invention, Hsp 90 activity is inhibited by applying a pharmacological agent. In further specific embodiments, the pharmacological agent reduces Hsp90 function, preferably resulting in unmasking genetic variation by manifesting pre-existing polymorphisms. The application of the agent either directly or indirectly results in detection of a phenotype or, alternatively, a genotype, associated with the polymorphism. The phenotype may be associated with cotyledon morphology, adult leaf morphology, hypocotyl morphology, root morphology, root hair morphology, and/or rosette morphology.

The pharmacological agent may be of any class of compound, so long as it inhibits Hsp90 activity. In one embodiment, the agent inhibits Hsp90 through direct contact. In specific embodiments, the contact is at a singular point. In other embodiments, the contact is through multiple and distinct contacts with residues in the protein. In further specific embodiments, the multiple and distinct contacts are with residues in the highly unusual, evolutionarily conserved nucleotide-binding pocket of the protein. In a specific embodiment, the nucleotide is ATP. In another embodiment, the agent inhibits Hsp90 synthesis.

In other embodiments, the pharmacological agent is inactivated, such as by a stimulus, including prolonged exposure to light, exposure to heat or cold, administration of an inactivating agent, and the like. In a specific embodiment, the agent is an ATP-competitive inhibitor of Hsp90. In specific embodiments of the present invention, the pharmacological agent is a benzoquinone ansamycin or a macrolactone. In another specific embodiment of the present invention, the pharmacological agent is geldanamycin or radicicol. In a further specific embodiment, the geldanamycin is 17allylamino-17-demethoxygeldanamycin (17-AAG). In an additional specific embodiment, the agent is herbimycin A, macbecin I, or quercetin.

II. Desirable Characteristics for Plants

It is possible that the plant of the present invention may have other properties or characteristics which are altered, in specific embodiments, in addition to being altered for the inhibition of Hsp90 activity. For instance, a plant may be used which already has improved pest protection qualities, resistance to herbicides, increased ability to tolerate environmental stress, resistance to disease, reduction in mycotoxin reduction and/or elimination, improvement in grain quality, improvement in agronomic characteristics, enhancement of nutrient utilization, conferring of male sterility, conferring of a negative selectable marker, introduction of non-protein-expressing sequences to affect plant phenotype, and so forth. The improvements may be through genetic engineering or by traditional breeding practices.

Thus, in certain embodiments of the invention, transformation of a recipient cell may be carried out with more than one exogenous (selected) gene. As used herein, an “exogenous coding region” or “selected coding region” is a coding region not normally found in the host genome in an identical context. By this, it is meant that the coding region may be isolated from a different species than that of the host genome, or alternatively, isolated from the host genome, but is operably linked to one or more regulatory regions which differ from those found in the unaltered, native gene. Two or more exogenous coding regions also can be supplied in a single transformation event using either distinct transgene-encoding vectors, or using a single vector incorporating two or more coding sequences. For example, plasmids bearing the bar and aroA expression units in either convergent, divergent, or colinear orientation, are considered to be particularly useful. Further preferred combinations are those of an insect resistance gene, such as a Bt gene, along with a protease inhibitor gene such as pinII, or the use of bar in combination with either of the above genes. Of course, any two or more transgenes of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired.

A. Herbicide Resistance

The DNA segments encoding phosphinothricin acetyltransferase (bar and pat), EPSP synthase encoding genes conferring resistance to glyphosate, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are examples of herbicide resistant genes for use in transformation. Other examples include the bar and pat genes, which code for the enzyme phosphinothricin acetyltransferase (PAT). Also, the enzyme 5-enolpyruvylshikimate 3-phosphate synthase (EPSP Synthase) is normally inhibited by the herbicide N-(phosphonomethyl)glycine (glyphosate), but genes are known that encode glyphosate-resistant EPSP synthase enzymes, such as the deh gene or the bxn gene.

B. Insect Resistance

Potential insect resistance genes that can be introduced include Bacillus thuringiensis crystal toxin genes or Bt genes (Watrud et al., 1985). Bt genes may provide resistance to lepidopteran or coleopteran pests such as European Corn Borer (ECB). It is contemplated that preferred Bt genes for use in the transformation protocols disclosed herein will be those in which the coding sequence has been modified to effect increased expression in plants, and more particularly, in maize. Means for preparing synthetic genes are well known in the art and are disclosed in, for example, U.S. Pat. No. 5,500,365 and U.S. Pat. No. 5,689,052, each of the disclosures of which are specifically incorporated herein by reference in their entirety. Examples of such modified Bt toxin genes include a synthetic Bt CryIA(b) gene (Perlak et al., 1991), and the synthetic CryIA(c) gene termed 1800b (PCT Application WO 95/06128).

Other examples include protease inhibitors, such as pinII (from tomato or potato), genes which encode inhibitors of the insect's digestive system, or those that encode enzymes or co-factors that facilitate the production of inhibitors, also may be useful. Additional examples also include genes encoding lectins, genes controlling the production of large or small polypeptides active against insects when introduced into the insect pests, (such as, e.g., lytic peptides, peptide hormones and toxins and venoms)

C. Environment or Stress Resistance

Improvement of a plants ability to tolerate various environmental stresses such as, but not limited to, drought, excess moisture, chilling, freezing, high temperature, salt, and oxidative stress, also can be effected through expression of novel genes. It is proposed that benefits may be realized in terms of increased resistance to freezing temperatures through the introduction of an “antifreeze” protein such as that of the Winter Flounder (Cutler et al., 1989) or synthetic gene derivatives thereof. Improved chilling tolerance also may be conferred through increased expression of glycerol-3-phosphate acetyltransferase in chloroplasts (Wolter et al., 1992). Resistance to oxidative stress (often exacerbated by conditions such as chilling temperatures in combination with high light intensities) can be conferred by expression of superoxide dismutase (Gupta et al., 1993), and may be improved by glutathione reductase (Bowler et al., 1992). Such strategies may allow for tolerance to freezing in newly emerged fields as well as extending later maturity higher yielding varieties to earlier relative maturity zones.

It is contemplated that the expression of novel genes that favorably effect plant water content, total water potential, osmotic potential, and turgor will enhance the ability of the plant to tolerate drought. As used herein, the terms “drought resistance” and “drought tolerance” are used to refer to a plants increased resistance or tolerance to stress induced by a reduction in water availability, as compared to normal circumstances, and the ability of the plant to function and survive in lower-water environments. In this aspect of the invention it is proposed, for example, that the expression of genes encoding for the biosynthesis of osmotically-active solutes, such as polyol compounds, may impart protection against drought. Within this class are genes encoding for mannitol-L-phosphate dehydrogenase (Lee and Saier, 1982) and trehalose-6-phosphate synthase (Kaasen et al., 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., 1992, 1993). Altered water utilization in transgenic corn producing mannitol also has been demonstrated (U.S. Pat. No. 5,780,709).

D. Disease Resistance

It is proposed that increased resistance to diseases may be realized through introduction of genes into plants, for example, into monocotyledonous plants such as maize. It is possible to produce resistance to diseases caused by viruses, bacteria, fungi and nematodes. It also is contemplated that control of mycotoxin producing organisms may be realized through expression of introduced genes.

Resistance to viruses may be produced through expression of novel genes. For example, it has been demonstrated that expression of a viral coat protein in a transgenic plant can impart resistance to infection of the plant by that virus and perhaps other closely related viruses (Cuozzo et al., 1988, Hemenway et al., 1988, Abel et al., 1986). It is contemplated that expression of antisense genes targeted at essential viral functions also may impart resistance to viruses. For example, an antisense gene targeted at the gene responsible for replication of viral nucleic acid may inhibit replication and lead to resistance to the virus. It is believed that interference with other viral functions through the use of antisense genes also may increase resistance to viruses. Similarly, ribozymes could be used in this context. Further, it is proposed that it may be possible to achieve resistance to viruses through other approaches, including, but not limited to the use of satellite viruses.

Increased resistance to diseases caused by bacteria and fungi also may be realized through introduction of novel genes. It is contemplated that genes encoding so-called “peptide antibiotics,” pathogenesis related (PR) proteins, toxin resistance, and proteins affecting host-pathogen interactions such as morphological characteristics will be useful. Peptide antibiotics are polypeptide sequences which are inhibitory to growth of bacteria and other microorganisms. For example, the classes of peptides referred to as cecropins and magainins inhibit growth of many species of bacteria and fungi. It is proposed that expression of PR proteins in monocotyledonous plants such as maize may be useful in conferring resistance to bacterial disease. These genes are induced following pathogen attack on a host plant and have been divided into at least five classes of proteins (Bol, Linthorst, and Comelissen, 1990). Included amongst the PR proteins are β-1, 3-glucanases, chitinases, and osmotin and other proteins that are believed to function in plant resistance to disease organisms. Other genes have been identified that have antifungal properties, e.g., UDA (stinging nettle lectin) and hevein (Broakaert et al., 1989; Barkai-Golan et al., 1978).

E. Mycotoxin Reduction/Elimination

Production of mycotoxins, including aflatoxin and fumonisin, by fungi associated with monocotyledonous plants such as maize is a significant factor in rendering the grain not useful. These fungal organisms do not cause disease symptoms and/or interfere with the growth of the plant, but they produce chemicals (mycotoxins) that are toxic to animals. It is contemplated that inhibition of the growth of these fungi would reduce the synthesis of these toxic substances and therefore reduce grain losses due to mycotoxin contamination. It also is proposed that it may be possible to introduce novel genes into monocotyledonous plants such as maize that would inhibit synthesis of the mycotoxin. Further, it is contemplated that expression of a novel gene which encodes an enzyme capable of rendering the mycotoxin nontoxic would be useful in order to achieve reduced mycotoxin contamination of grain. The result of any of the above mechanisms would be a reduced presence of mycotoxins on grain.

F. Grain Composition or Quality

Genes may be introduced into monocotyledonous plants, particularly commercially important cereals such as maize, to improve the grain for which the cereal is primarily grown. A wide range of novel transgenic plants produced in this manner may be envisioned depending on the particular end use of the grain.

The largest use of maize grain is for feed or food. Introduction of genes that alter the composition of the grain may greatly enhance the feed or food value. The primary components of maize grain are starch, protein, and oil. Each of these primary components of maize grain may be improved by altering its level or composition. Several examples may be mentioned for illustrative purposes, but in no way provide an exhaustive list of possibilities.

For example, the protein of cereal grains is deficient in several amino acids, such as lysine, methionine, tryptophan, threonine, valine, arginine, and histidine. Some amino acids become limiting only after corn is supplemented with other inputs for feed formulations. For example, when corn is supplemented with soybean meal to meet lysine requirements methionine becomes limiting. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

The introduction of genes that alter the oil content of the grain may be of value. Increases in oil content may result in increases in metabolizable-energy-content and density of the seeds for use in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, β-ketoacyl-ACP synthase, plus other well known fatty acid biosynthetic activities. Other possibilities are genes that encode proteins that do not possess enzymatic activity such as acyl carrier protein. Genes may be introduced that alter the balance of fatty acids present in the oil providing a more healthful or nutritive feedstuff. The introduced DNA also may encode sequences that block expression of enzymes involved in fatty acid biosynthesis, altering the proportions of fatty acids present in the grain such as described below. Some other examples of genes specifically contemplated by the inventors for use in creating transgenic plants with altered oil composition traits include 2-acetyltransferase, oleosin, pyruvate dehydrogenase complex, acetyl CoA synthetase, ATP citrate lyase, ADP-glucose pyrophosphorylase and genes of the carnitine-CoA-acetyl-CoA shuttles. It is anticipated that expression of genes related to oil biosynthesis will be targeted to the plastid, using a plastid transit peptide sequence and preferably expressed in the seed embryo.

Feed or food comprising primarily maize or other cereal grains possesses insufficient quantities of vitamins and must be supplemented to provide adequate nutritive value. Introduction of genes that enhance vitamin biosynthesis in seeds may be envisioned including, for example, vitamins A, E, B₁₂, choline, and the like. Maize grain also does not possess sufficient mineral content for optimal nutritive value. Genes that affect the accumulation or availability of compounds containing phosphorus, sulfur, calcium, manganese, zinc, and iron among others would be valuable. An example may be the introduction of a gene that reduced phytic acid production or encoded the enzyme phytase which enhances phytic acid breakdown. These genes would increase levels of available phosphate in the diet, reducing the need for supplementation with mineral phosphate.

In addition to direct improvements in feed or food value, genes also may be introduced which improve the processing of corn and improve the value of the products resulting from the processing. The primary method of processing corn is via wetmilling. Maize may be improved though the expression of novel genes that increase the efficiency and reduce the cost of processing such as by decreasing steeping time. Improving the value of wetmilling products may include altering the quantity or quality of starch, oil, corn gluten meal, or the components of corn gluten feed.

In addition, it may further be considered that the corn plant be used for the production or manufacturing of useful biological compounds that were either not produced at all, or not produced at the same level, in the corn plant previously. The novel corn plants producing these compounds are made possible by the introduction and expression of genes by corn transformation methods. The vast array of possibilities include but are not limited to any biological compound which is presently produced by any organism such as proteins, nucleic acids, primary and intermediary metabolites, carbohydrate polymers, etc. The compounds may be produced by the plant, extracted upon harvest and/or processing, and used for any presently recognized useful purpose such as pharmaceuticals, fragrances, and industrial enzymes to name a few.

Further possibilities, to exemplify the range of grain traits or properties potentially encoded by introduced genes in transgenic plants, include grain with less breakage susceptibility for export purposes or larger grit size when processed by dry milling through introduction of genes that enhance γ-zein synthesis, popcorn with improved popping quality and expansion volume through genes that increase pericarp thickness, corn with whiter grain for food uses though introduction of genes that effectively block expression of enzymes involved in pigment production pathways, and improved quality of alcoholic beverages or sweet corn through introduction of genes which affect flavor such as the shrunken 1 gene (encoding sucrose synthase) or shrunken 2 gene (encoding ADPG pyrophosphorylase) for sweet corn.

G. Plant Agronomic Characteristics

Two of the factors determining where crop plants can be grown are the average daily temperature during the growing season and the length of time between frosts. Within the areas where it is possible to grow a particular crop, there are varying limitations on the maximal time it is allowed to grow to maturity and be harvested. For example, maize to be grown in a particular area is selected for its ability to mature and dry down to harvestable moisture content within the required period of time with maximum possible yield. Therefore, corn of varying maturities is developed for different growing locations. Apart from the need to dry down sufficiently to permit harvest, it is desirable to have maximal drying take place in the field to minimize the amount of energy required for additional drying post-harvest. Also, the more readily the grain can dry down, the more time there is available for growth and kernel fill. It is considered that genes that influence maturity and/or dry down can be identified and introduced into corn or other plants using transformation techniques to create new varieties adapted to different growing locations or the same growing location, but having improved yield to moisture ratio at harvest. Expression of genes that are involved in regulation of plant development may be especially useful, e.g., the liguleless and rough sheath genes that have been identified in corn.

It is contemplated that genes may be introduced into plants that would improve standability and other plant growth characteristics. Expression of novel genes in maize which confer stronger stalks, improved root systems, or prevent or reduce ear droppage would be of great value to the farmer. It is proposed that introduction and expression of genes that increase the total amount of photoassimilate available by, for example, increasing light distribution and/or interception would be advantageous. In addition, the expression of genes that increase the efficiency of photosynthesis and/or the leaf canopy would further increase gains in productivity. It is contemplated that expression of a phytochrome gene in corn may be advantageous. Expression of such a gene may reduce apical dominance, confer semidwarfism on a plant, and increase shade tolerance (U.S. Pat. No. 5,268,526). Such approaches would allow for increased plant populations in the field.

H. Nutrient Utilization

The ability to utilize available nutrients may be a limiting factor in growth of monocotyledonous plants such as maize. It is proposed that it would be possible to alter nutrient uptake, tolerate pH extremes, mobilization through the plant, storage pools, and availability for metabolic activities by the introduction of novel genes. These modifications would allow a plant such as maize to more efficiently utilize available nutrients. It is contemplated that an increase in the activity of, for example, an enzyme that is normally present in the plant and involved in nutrient utilization would increase the availability of a nutrient. An example of such an enzyme would be phytase. It further is contemplated that enhanced nitrogen utilization by a plant is desirable. Expression of a glutamate dehydrogenase gene in corn, e.g., E. coli gdhA genes, may lead to increased fixation of nitrogen in organic compounds. Furthermore, expression of gdhA in corn may lead to enhanced resistance to the herbicide glufosinate by incorporation of excess ammonia into glutamate, thereby detoxifying the ammonia. It also is contemplated that expression of a novel gene may make a nutrient source available that was previously not accessible, e.g., an enzyme that releases a component of nutrient value from a more complex molecule, perhaps a macromolecule.

I. Male Sterility

Male sterility is useful in the production of hybrid seed. It is proposed that male sterility may be produced through expression of novel genes. For example, it has been shown that expression of genes that encode proteins that interfere with development of the male inflorescence and/or gametophyte result in male sterility. Chimeric ribonuclease genes that express in the anthers of transgenic tobacco and oilseed rape have been demonstrated to lead to male sterility (Mariani et al., 1990).

A number of mutations have been discovered in maize that confer cytoplasmic male sterility. One mutation in particular, referred to as T cytoplasm, also correlates with sensitivity to Southern corn leaf blight. A DNA sequence, designated TURF-13 (Levings, 1990), was identified that correlates with T cytoplasm. It is proposed that it would be possible through the introduction of TURF-13 via transformation, to separate male sterility from disease sensitivity. As it is necessary to be able to restore male fertility for breeding purposes and for grain production, it is proposed that genes encoding restoration of male fertility also may be introduced.

J. Negative Selectable Markers

Introduction of genes encoding traits that can be selected against may be useful for eliminating undesirable linked genes. It is contemplated that when two or more genes are introduced together by cotransformation that the genes will be linked together on the host chromosome. For example, a gene encoding Bt that confers insect resistance on the plant may be introduced into a plant together with a bar gene that is useful as a selectable marker and confers resistance to the herbicide Liberty® on the plant. However, it may not be desirable to have an insect resistant plant that also is resistant to the herbicide Liberty®. It is proposed that one also could introduce an antisense bar coding region that is expressed in those tissues where one does not want expression of the bar gene product, e.g., in whole plant parts. Hence, although the bar gene is expressed and is useful as a selectable marker, it is not useful to confer herbicide resistance on the whole plant. The bar antisense gene is a negative selectable marker.

It also is contemplated that negative selection is necessary in order to screen a population of transformants for rare homologous recombinants generated through gene targeting. For example, a homologous recombinant may be identified through the inactivation of a gene that was previously expressed in that cell. The antisense construct for neomycin phosphotransferase II (NPT II) has been investigated as a negative selectable marker in tobacco (Nicotiana tabacum) and Arabidopsis thaliana (Xiang. and Guerra, 1993). In this example, both sense and antisense NPT II genes are introduced into a plant through transformation and the resultant plants are sensitive to the antibiotic kanamycin. An introduced gene that integrates into the host cell chromosome at the site of the antisense NPT II gene, and inactivates the antisense gene, will make the plant resistant to kanamycin and other aminoglycoside antibiotics. Therefore, rare, site-specific recombinants may be identified by screening for antibiotic resistance. Similarly, any gene, native to the plant or introduced through transformation, that when inactivated confers resistance to a compound, may be useful as a negative selectable marker.

It is contemplated that negative selectable markers also may be useful in other ways, such as to construct transgenic lines in which one could select for transposition to unlinked sites or to construct transposon tagging lines.

K. Non-Protein-Expressing Sequences

DNA may be introduced into plants for the purpose of expressing RNA transcripts that function to affect plant phenotype yet are not translated into protein. Two examples are antisense RNA and RNA with ribozyme activity. Both may serve possible functions in reducing or eliminating expression of native or introduced plant genes. However, DNA need not be expressed to effect the phenotype of a plant. Examples include antisense RNA, ribozymes, induction of gene silencing, and non-RNA-expressing sequences (such as transposable elements).

III. Methods for Plant Transformation

Although one advantage to utilizing the present invention is to circumvent creating genetically modified organisms in order to acquire desirable plant traits, in particular embodiments of the present invention it is desirable to transform a plant, thereby generating a transgenic plant. Suitable methods for plant transformation for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, maize cells as well as those of virtually any other plant species may be stably transformed, and these cells developed into transgenic plants. In certain embodiments, acceleration methods are preferred and include, for example, microprojectile bombardment and the like.

A. Electroporation

Where one wishes to introduce DNA by means of electroporation, it is contemplated that the method of Krzyzek et al. (U.S. Pat. No. 5,384,253, incorporated herein by reference in its entirety) will be particularly advantageous. In this method, certain cell wall-degrading enzymes, such as pectin-degrading enzymes, are employed to render the target recipient cells more susceptible to transformation by electroporation than untreated cells. Alternatively, recipient cells are made more susceptible to transformation by mechanical wounding.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

B. Microprojectile Bombardment

A preferred method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

C. Agrobacterium-mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; Zhang et al., 1997; U.S. Pat. No. 5,591,616, specifically incorporated herein by reference in its entirety), wheat (McCormac et al., 1998), barley (Tingay et al., 1997; McCormac et al., 1998), and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

D. Other Transformation Methods

Transformation of plant protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Fujimara et al., 1985; Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cell are punctured. This technique has been used successfully with, for example, the monocot cereals maize (PCT Application WO 95/06128, specifically incorporated herein by reference in its entirety; Thompson, 1995) and rice (Nagatani, 1997).

IV. Plant Transformation Constructs

The construction of vectors which may be employed in conjunction with plant transformation techniques according to the invention will be known to those of skill of the art in light of the present disclosure (see for example, Sambrook et al., 1989; Gelvin et al., 1990). The techniques of the current invention are thus not limited to any particular DNA sequences in conjunction with a desired promoter of the invention. For example, the desired promoter alone could be transformed into a plant with the goal of enhancing or altering the expression of one or more genes in the host genome.

One important use of the sequences of the invention will be in directing the expression of a selected coding region which encodes a particular protein or polypeptide product. However, the selected coding regions also may be non-expressible DNA segments, e.g., transposons such as Ds that do not direct their own transposition. The inventors also contemplate that, where both an expressible gene that is not necessarily a marker gene is employed in combination with a marker gene, one may employ the separate genes on either the same or different DNA segments for transformation. In the latter case, the different vectors are delivered concurrently to recipient cells to maximize cotransformation.

The choice of the particular selected coding regions used in accordance with the desired promoter for transformation of recipient cells will often depend on the purpose of the transformation. One of the major purposes of transformation of crop plants is to add commercially desirable, agronomically important traits to the plant. Such traits include, but are not limited to, herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress, or oxidative stress; increased yields; food content and makeup; physical appearance; male sterility; drydown; standability; prolificacy; starch properties; oil quantity and quality, and the like.

In certain embodiments, the present inventors contemplate the transformation of a recipient cell with more than transformation construct. Two or more transgenes can be created in a single transformation event using either distinct selected-protein encoding vectors, or using a single vector incorporating two or more gene coding sequences. Of course, any two or more transgenes of any description, such as those conferring, for example, herbicide, insect, disease (viral, bacterial, fungal, nematode) or drought resistance, male sterility, drydown, standability, prolificacy, starch properties, oil quantity and quality, or those increasing yield or nutritional quality may be employed as desired.

In other embodiments of the invention, it is contemplated that one may wish to employ replication-competent viral vectors for plant transformation. Such vectors include, for example, wheat dwarf virus (WDV) “shuttle” vectors, such as pW1-111 and PW1-GUS (Ugaki et al., 1991). These vectors are capable of autonomous replication in maize cells as well as E. coli, and as such may provide increased sensitivity for detecting DNA delivered to transgenic cells. A replicating vector also may be useful for delivery of genes flanked by DNA sequences from transposable elements such as Ac, Ds, or Mu. It has been proposed that transposition of these elements within the maize genome requires DNA replication (Laufs et al., 1990). It also is contemplated that transposable elements would be useful for introducing DNA fragments lacking elements necessary for selection and maintenance of the plasmid vector in bacteria, e.g., antibiotic resistance genes and origins of DNA replication. It also is proposed that use of a transposable element such as Ac, Ds, or Mu would actively promote integration of the desired DNA and hence increase the frequency of stably transformed cells.

It further is contemplated that one may wish to co-transform plants or plant cells with 2 or more vectors. Co-transformation may be achieved using a vector containing the marker and another gene or genes of interest. Alternatively, different vectors, e.g., plasmids, may contain the different genes of interest, and the plasmids may be concurrently delivered to the recipient cells. Using this method, the assumption is made that a certain percentage of cells in which the marker has been introduced, also have received the other gene(s) of interest. Thus, not all cells selected by means of the marker, will express the other proteins of interest which had been presented to the cells concurrently.

Vectors used for plant transformation may include, for example, plasmids, cosmids, YACs (yeast artificial chromosomes), BACs (bacterial artificial chromosomes) or any other suitable cloning system. It is contemplated that utilization of cloning systems with large insert capacities will allow introduction of large DNA sequences comprising more than one selected gene. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes which have been isolated from such vectors. DNA segments used for transforming plant cells will, of course, generally comprise the cDNA, gene or genes which one desires to introduced into and have expressed in the host cells. These DNA segments can further include, in addition to a desired promoter, structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Plant Promoters

Promoters which are useful for plant transgene expression include those that are inducible, viral, synthetic, constitutive as described (Poszkowski et al., 1989; Odell et al., 1985), temporally regulated, spatially regulated, and spatio-temporally regulated (Chau et al., 1989).

A number of plant promoters have been described with various expression characteristics. Examples of some constitutive promoters which have been described include the rice actin 1 (Wang et al., 1992; U.S. Pat. No. 5,641,876), CaMV 35S (Odell et al., 1985), CaMV 19S (Lawton et al., 1987), nos (Ebert et al., 1987), Adh (Walker et al., 1987), sucrose synthase (Yang & Russell, 1990).

Examples of tissue specific promoters which have been described include the lectin (Vodkin et al., 1983; Lindstrom et al., 1990), corn alcohol dehydrogenase 1 (Vogel et al., 1989; Dennis et al., 1984), corn light harvesting complex (Simpson, 1986; Bansal et al., 1992), corn heat shock protein (Odell et al., 1985; Rochester et al., 1986), pea small subunit RuBP carboxylase (Poulsen et al., 1986; Cashmore et al., 1983), Ti plasmid mannopine synthase (Langridge et al., 1989), Ti plasmid nopaline synthase (Langridge et al., 1989), petunia chalcone isomerase (Van Tunen et al., 1988), bean glycine rich protein 1 (Keller et al., 1989), truncated CaMV 35s (Odell et al., 1985), potato patatin (Wenzler et al., 1989), root cell (Conkling et al., 1990), maize zein (Reina et al., 1990; Kriz et al., 1987; Wandelt and Feix, 1989; Langridge and Feix, 1983; Reina et al., 1990), globulin-1 (Belanger and Kriz et al., 1991), α-tubulin, cab (Sullivan et al., 1989), PEPCase (Hudspeth & Grula, 1989), R gene complex-associated promoters (Chandler et al., 1989), and chalcone synthase promoters (Franken et al., 1991).

Inducible promoters which have been described include ABA- and turgor-inducible promoters, the promoter of the auxin-binding protein gene (Schwob et al., 1993), the UDP glucose flavonoid glycosyl-transferase gene promoter (Ralston et al., 1988); the MPI proteinase inhibitor promoter (Cordero et al, 1994), and the glyceraldehyde-3-phosphate dehydrogenase gene promoter (Kohler et al., 1995; Quigley et al., 1989; Martinez et al., 1989).

A class of genes which are expressed in an inducible manner are glycine-rich proteins GRPs). GRPs are a class of proteins characterized by their high content of glycine residues, which often occur in repetitive blocks (Goddemeier et al., 1998). Many GRPs are thought to be structural wall proteins or RNA-binding proteins (Mar Alba et al., 1994). Genes encoding glycine rich proteins have been described, for example, from maize (Didierjean et al., 1992; Baysdorfer, Genbank Accession No. AF034945) sorghum (Cretin and Puigdomenech, 1990), and rice (Lee et al., Genbank Accession No. AF00941 1).

B. Plant Promoter Derivatives

Derivatives of plant promotes may be generated by a variety of means. Mutagenesis may be carried out at random and the mutagenized sequences screened for function in a trial-by-error procedure. Alternatively, particular sequences which provide the promoter with desirable expression characteristics could be identified and these or similar sequences introduced into other related or non-related sequences via mutation. Similarly, non-essential elements may be deleted without significantly altering the function of the elements. It further is contemplated that one could mutagenize these sequences in order to enhance their utility in expressing transgenes in a particular species, for example, in maize.

The means for mutagenizing a DNA segment encoding a desired promoter sequence of the current invention are well-known to those of skill in the art. Mutagenesis may be performed in accordance with any of the techniques known in the art, such as, and not limited to, synthesizing an oligonucleotide having one or more mutations within the sequence of a particular regulatory region. In particular, site-specific mutagenesis is a technique useful in the preparation of promoter mutants, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the Ml 3 phage. These phage are readily commercially available and their use is generally well known to those skilled in the art. Double stranded plasmids also are routinely employed in site directed mutagenesis which eliminates the step of transferring the gene of interest from a plasmid to a phage.

Site-directed mutagenesis in accordance herewith typically is performed by first obtaining a single-stranded vector or melting apart of two strands of a double stranded vector which includes within its sequence a DNA sequence which encodes the maize GRP promoter. An oligonucleotide primer bearing the desired mutated sequence is prepared, generally synthetically. This primer is then annealed with the single-stranded vector, and subjected to DNA polymerizing enzymes such as the E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and cells are selected which include recombinant vectors bearing the mutated sequence arrangement. Vector DNA can then be isolated from these cells and used for plant transformation. A genetic selection scheme was devised by Kunkel et al. (1987) to enrich for clones incorporating mutagenic oligonucleotides. Alternatively, the use of PCR™ with commercially available thermostable enzymes such as Taq polymerase may be used to incorporate a mutagenic oligonucleotide primer into an amplified DNA fragment that can then be cloned into an appropriate cloning or expression vector. The PCR™-mediated mutagenesis procedures of Tomic et al. (1990) and Upender et al. (1995) provide two examples of such protocols. A PCR™ employing a thermostable ligase in addition to a thermostable polymerase also may be used to incorporate a phosphorylated mutagenic oligonucleotide into an amplified DNA fragment that may then be cloned into an appropriate cloning or expression vector.

The preparation of sequence variants of the selected promoter DNA segments using site-directed mutagenesis is provided as a means of producing potentially useful species and is not meant to be limiting as there are other ways in which sequence variants of DNA sequences may be obtained. For example, recombinant vectors encoding the desired promoter sequence may be treated with mutagenic agents, such as hydroxylamine, to obtain sequence variants.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” also is intended to refer to a process that involves the template-dependent extension of a primer molecule. The term template-dependent process refers to nucleic acid synthesis of an RNA or a DNA molecule wherein the sequence of the newly synthesized strand of nucleic acid is dictated by the well-known rules of complementary base pairing (see, for example, Watson and Ramstad, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety. A number of template dependent processes are available to amplify the target sequences of interest present in a sample, such methods being well known in the art and specifically disclosed herein below.

One efficient, targeted means for preparing mutagenized promoters or enhancers relies upon the identification of putative regulatory elements within the target sequence. This can be initiated by comparison with, for example, promoter sequences known to be expressed in a similar manner. Sequences which are shared among elements with similar functions or expression patterns are likely candidates for the binding of transcription factors and are thus likely elements which confer expression patterns. Confirmation of these putative regulatory elements can be achieved by deletion analysis of each putative regulatory region followed by functional analysis of each deletion construct by assay of a reporter gene which is functionally attached to each construct. As such, once a starting promoter or intron sequence is provided, any of a number of different functional deletion mutants of the starting sequence could be readily prepared.

As indicated above, deletion mutants of a desired promoter also could be randomly prepared and then assayed. With this strategy, a series of constructs are prepared, each containing a different portion of the clone (a subclone), and these constructs are then screened for activity. A suitable means for screening for activity is to attach a deleted promoter construct to a selectable or screenable marker, and to isolate only those cells expressing the marker protein. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous protein.

C. Regulatory Elements

Constructs prepared in accordance with the current invention will include a desired promoter or a derivative thereof. However, these sequences may be used in the preparation of transformation constructs which comprise a wide variety of other elements. One such application in accordance with the instant invention will be the preparation of transformation constructs comprising the desired promoter operably linked to a selected coding region. By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers which could be used in accordance with the invention include elements from the CaMV 35S promoter, octopine synthase genes (Ellis et al., 1987), the maize alcohol dehydrogenase gene, the maize shrunken 1 gene and promoters from non-plant eukaryotes (e.g., yeast; Ma et al., 1988).

Where an enhancer is used in conjunction with a desired promoter for the expression of a selected protein, it is believed that it will be preferred to place the enhancer between the promoter and the start codon of the selected coding region. However, one also could use a different arrangement of the enhancer relative to other sequences and still realize the beneficial properties conferred by the enhancer. For example, the enhancer could be placed 5′ of the promoter region, within the promoter region, within the coding sequence (including within any other intron sequences which may be present), or 3′ of the coding region.

In addition to introns with enhancing activity, other types of elements can influence gene expression. For example, untranslated leader sequences have been made to predict optimum or sub-optimum sequences and generate “consensus” and preferred leader sequences (Joshi, 1987). Preferred leader sequences are contemplated to include those which have sequences predicted to direct optimum expression of the attached coding region, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants, and in maize in particular, will be most preferred.

Specifically contemplated for use in accordance with the present invention are vectors which include the ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). It is proposed that the use of an enhancer element, such as the ocs element and particularly multiple copies of the element, may be used to increase the level of transcription from adjacent promoters when applied in the context of monocot transformation.

Ultimately, the most desirable DNA segments for introduction into a plant genome may be homologous genes or gene families which encode a desired trait, and which are introduced under the control of the desired promoter. The tissue-specific expression profile of the desired promoter will be of particular benefit in the expression of transgenes in plants. For example, it is envisioned that a particular use of the present invention may be the production of transformants comprising a transgene which is expressed in a tissue-specific manner, whereby the expression is enhanced by an actin 1 or actin 2 intron. For example, insect resistant protein may be expressed specifically in the roots which are targets for a number of pests including nematodes and the corn root worm.

It also is contemplated that expression of one or more transgenes may be obtained in all tissues but roots by introducing a constitutively expressed gene (all tissues) in combination with an antisense gene that is expressed only by the desired promoter. Therefore, expression of an antisense transcript encoded by the constitutive promoter would prevent accumulation of the respective protein encoded by the sense transcript. Similarly, antisense technology could be used to achieve temporally-specific or inducible expression of a transgene encoded by a desired promoter.

It also is contemplated that it may be useful to target DNA within a cell. For example, it may be useful to target introduced DNA to the nucleus as this may increase the frequency of transformation. Within the nucleus itself, it would be useful to target a gene in order to achieve site specific integration. For example, it would be useful to have a gene introduced through transformation replace an existing gene in the cell.

D. Terminators

Transformation constructs prepared in accordance with the invention will typically include a 3′ end DNA sequence that acts as a signal to terminate transcription and allow for the poly-adenylation of the mRNA produced by coding sequences operably linked to the maize GRP promoter. One type of terminator which may be used is a terminator from a gene encoding the small subunit of a ribulose-1,5-bisphosphate carboxylase-oxygenase (rbcS), and more specifically, from a rice rbcS gene. Where a 3′ end other than an rbcS terminator is used in accordance with the invention, the most preferred 3′ ends are contemplated to be those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as Adh intron (Callis et al., 1987), sucrose synthase intron (Vasil et al., 1989) or TMV omega element (Gallie, et al., 1989), may further be included where desired. Alternatively, one also could use a gamma coixin, oleosin 3 or other terminator from the genus Coix.

E. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

A particular example of such a use concerns the direction of a protein conferring herbicide resistance, such as a mutant EPSPS protein, to a particular organelle such as the chloroplast rather than to the cytoplasm. This is exemplified by the use of the rbcS transit peptide, the chloroplast transit peptide described in U.S. Pat. No. 5,728,925, or the optimized transit peptide described in U.S. Pat. No. 5,510,471, which confers plastid-specific targeting of proteins. In addition, it may be desirable to target certain genes responsible for male sterility to the mitochondria, or to target certain genes for resistance to phytopathogenic organisms to the extracellular spaces, or to target proteins to the vacuole. A further use concerns the direction of enzymes involved in amino acid biosynthesis or oil synthesis to the plastid. Such enzymes include dihydrodipicolinic acid synthase which may contribute to increasing lysine content of a feed.

F. Marker Genes

One application of a desired promoter for the current invention will be in the expression of marker proteins. By employing a selectable or screenable marker gene as, or in addition to, the gene of interest, one can provide or enhance the ability to identify transformants. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker genes are known to the art and can be employed in the practice of the invention.

Included within the terms selectable or screenable marker genes also are genes which encode a “secretable marker” whose secretion can be detected as a means of identifying or selecting for transformed cells. Examples include marker genes which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). The use of maize HPRG (Steifel et al., 1990) is preferred, as this molecule is well characterized in terms of molecular biology, expression and protein structure. However, any one of a variety of extensins and/or glycine-rich wall proteins (Keller et al., 1989) could be modified by the addition of an antigenic site to create a screenable marker.

One exemplary embodiment of a secretable screenable marker concerns the use of a HPRG sequence modified to include a 15 residue epitope from the pro-region of murine interleukin-1-β (IL-1-β). However, virtually any detectable epitope may be employed in such embodiments, as selected from the extremely wide variety of antigen:antibody combinations known to those of skill in the art. The unique extracellular epitope, whether derived from IL-1β or any other protein or epitopic substance, can then be straightforwardly detected using antibody labeling in conjunction with chromogenic or fluorescent adjuncts.

G. Selectable Markers

Many selectable marker coding regions may be used in connection with the ZMGRP promoter of the present invention including, but not limited to, neo (Potrykus et al., 1985) which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154,204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan. Where a mutant EPSP synthase is employed, additional benefit may be realized through the incorporation of a suitable chloroplast transit peptide, CTP (U.S. Pat. No. 5,188,642) or OTP (U.S. Pat. No. 5,633,448) and use of a modified maize EPSPS (PCT Application WO 97/04103).

An illustrative embodiment of selectable markers capable of being used in systems to select transformants are the enzyme phosphinothricin acetyltransferase, such as bar from Streptomyces hygroscopicus or pat from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Where one desires to employ bialaphos resistance in the practice of the invention, the inventor has discovered that particularly useful genes for this purpose are the bar or pat genes obtainable from species of Streptomyces (e.g., ATCC No. 21,705). The cloning of the bar gene has been described (Murakami et al., 1986; Thompson et al., 1987) as has the use of the bar gene in the context of plants (De Block et al., 1987; De Block et al., 1989; U.S. Pat. No. 5,550,318).

H. Screenable Markers

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an a-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloffet al., 1997; Reichel et al., 1996; Tian et al., 1997; WO 97/41228).

Genes from the maize R gene complex are contemplated to be particularly useful as screenable markers. The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bzl and Bz2), but carries a recessive allele at the R locus, transformation of any cell from that line with R will result in red pigment formation. Exemplary lines include Wisconsin 22 which contains the rg-Stadler allele and TRI 12, a K55 derivative which is r-g, b, P1. Alternatively, any genotype of maize can be utilized if the C1 and R alleles are introduced together.

It further is proposed that R gene regulatory regions may be employed in chimeric constructs in order to provide mechanisms for controlling the expression of chimeric genes. More diversity of phenotypic expression is known at the R locus than at any other locus (Coe et al., 1988). It is contemplated that regulatory regions obtained from regions 5′ to the structural R gene would be valuable in directing the expression of genes for, e.g., insect resistance, herbicide tolerance or other protein coding regions. For the purposes of the present invention, it is believed that any of the various R gene family members may be successfully employed (e.g., P, S, Lc, etc.). However, the most preferred will generally be Sn (particularly Sn:bol3). Sn is a dominant member of the R gene complex and is functionally similar to the R and B loci in that Sn controls the tissue specific deposition of anthocyanin pigments in certain seedling and plant cells, therefore, its phenotype is similar to R.

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light. Where use of a screenable marker gene such as lux or GFP is desired, the inventors contemplated that benefit may be realized by creating a gene fusion between the screenable marker gene and a selectable marker gene, for example, a GFP-NPTII gene fusion. This could allow, for example, selection of transformed cells followed by screening of transgenic plants or seeds.

V. Production and Characterization of Stably Transformed Plants

After effecting delivery of exogenous DNA to recipient cells, the next steps generally concern identifying the transformed cells for further culturing and plant regeneration. As mentioned herein, in order to improve the ability to identify transformants, one may desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS, which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, bombarded tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

It further is contemplated that the herbicide DALAPON, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (Buchanan-Wollaston et al., 1992; U.S. patent application No. 08/113,561, filed Aug. 25, 1993; U.S. Pat. No. 5,508,468; and U.S. Pat. No. 5,508,468; each of the disclosures of which is specifically incorporated herein by reference in its entirety).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468; and U.S. patent application Ser. No. 08/604,789.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. A preferred growth regulator for such purposes is dicamba or 2,4-D. However, other growth regulators may be employed, including NAA, NAA+2,4-D or perhaps even picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber at about 85% relative humidity, 600 ppm CO₂, and 25-250 microeinsteins m⁻² s⁻¹ of light. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing.

Note, however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

Progeny may be recovered from transformed plants and tested for expression of the exogenous expressible gene by localized application of an appropriate substrate to plant parts such as leaves. In the case of bar transformed plants, it was found that transformed parental plants (R_{O}) and their progeny of any generation tested exhibited no bialaphos-related necrosis after localized application of the herbicide Basta to leaves, if there was functional PAT activity in the plants as assessed by an in vitro enzymatic assay. All PAT positive progeny tested contained bar, confirming that the presence of the enzyme and the resistance to bialaphos were associated with the transmission through the germline of the marker gene.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

1. DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR™). Using this technique discreet fragments of DNA are amplified and detected by gel electrophoresis. This type of analysis permits one to determine whether a gene is present in a stable transformant, but does not prove integration of the introduced gene into the host cell genome. It is the experience of the inventor, however, that DNA has been integrated into the genome of all transformants that demonstrate the presence of the gene through PCR™ analysis. In addition, it is not possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

2. Gene Expression

While Southern blotting and PCR™ may be used to detect the gene(s) in question, they do not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

VI. Plant Breeding

In addition to direct transformation of a particular plant genotype with a construct prepared as described herein, transgenic plants or other desirable plants may be made by crossing a plant having a construct of the invention to a second plant lacking the construct. For example, a selected DNA comprising a desirable trait can be introduced into a particular plant variety by crossing, without the need for ever directly transforming a plant of that given variety. Therefore, the current invention not only encompasses a plant directly regenerated from cells which have been transformed in accordance with the current invention, but also the progeny of such plants. As used herein the term “progeny” denotes the offspring of any generation of a parent plant prepared in accordance with the instant invention, wherein the progeny comprises a construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking said desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring said desired gene, DNA sequence or element from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking said desired DNA sequence may be referred to as an unconverted genotype, line, inbred, or hybrid.

Backcrossing can be used to improve a starting plant. Backcrossing transfers a specific desirable trait from a plant with one genetic background to another plant having a different genetic background which lacks that trait. This can be accomplished, for example, by first crossing a superior variety (for example, an inbred line) (recurrent parent) to a donor variety (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred, but are like the superior parent for most or almost all other genes. The last backcross generation would be selfed to give progeny which are pure breeding for the gene(s) being transferred, i.e. one or more transformation events.

Therefore, through a series a breeding manipulations, a selected transgene may be moved from one line into an entirely different line without the need for further recombinant manipulation. Transgenes are valuable in that they typically behave genetically as any other gene and can be manipulated by breeding techniques in a manner identical to any other corn gene. Therefore, one may produce inbred plants which are true breeding for one or more transgenes. By crossing different inbred plants, one may produce a large number of different hybrids with different combinations of transgenes. In this way, plants may be produced which have the desirable agronomic properties frequently associated with hybrids (“hybrid vigor”), as well as the desirable characteristics imparted by one or more transgene(s).

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

In the process of marker assisted breeding, DNA sequences are used to follow desirable agronomic traits in the process of plant breeding (Tanksley et al., 1989). Marker assisted breeding may be undertaken as follows. Seed of plants with the desired trait are planted in soil in the greenhouse or in the field. Leaf tissue is harvested from the plant for preparation of DNA at any point in growth at which approximately one gram of leaf tissue can be removed from the plant without compromising the viability of the plant. Genomic DNA is isolated using a procedure, such as one modified from Shure et al. (1983). In the technique of Shure et al. (1983), approximately one gram of leaf tissue from a seedling is lypholyzed overnight in 15 ml polypropylene tubes. Freeze-dried tissue is ground to a powder in the tube using a glass rod. Powdered tissue is mixed thoroughly with 3 ml extraction buffer (7.0 urea, 0.35 M NaCI, 0.05 M Tris-HCI pH 8.0, 0.01 M EDTA, 1% sarcosine). Tissue/buffer homogenate is extracted with 3 ml phenol/chloroform. The aqueous phase is separated by centrifugation, and precipitated twice using 1/10 volume of 4.4 M ammonium acetate pH 5.2, and an equal volume of isopropanol. The precipitate is washed with 75% ethanol and resuspended in 100-500 μl TE (0.01 M Tris-HCI, 0.001 M EDTA, pH 8.0).

Genomic DNA is then digested with a 3-fold excess of restriction enzymes, electrophoresed through 0.8% agarose (FMC), and transferred (Southern, 1975) to Nytran (Schleicher and Schuell) using 10× SCP (20 SCP: 2M NaCI, 0.6 M disodium phosphate, 0.02 M disodium EDTA). The filters are prehybridized in 6× SCP, 10% dextran sulfate, 2% sarcosine, and 500 μg/ml denatured salmon sperm DNA and ³²P-labeled probe generated by random priming (Feinberg & Vogelstein, 1983). Hybridized filters are washed in 2× SCP, 1% SDS at 65° C. for 30 minutes and visualized by autoradiography using Kodak XAR5 film. Genetic polymorphisms which are genetically linked to traits of interest are thereby used to predict the presence or absence of the traits of interest.

Those of skill in the art will recognize that there are many different ways to isolate DNA from plant tissues and that there are many different protocols for Southern hybridization that will produce identical results. Those of skill in the art will recognize that a Southern blot can be stripped of radioactive probe following autoradiography and re-probed with a different probe. In this manner one may identify each of the various transgenes that are present in the plant. Further, one of skill in the art will recognize that any type of genetic marker which is polymorphic at the region(s) of interest may be used for the purpose of identifying the relative presence or absence of a trait, and that such information may be used for marker assisted breeding.

Each lane of a Southern blot represents DNA isolated from one plant. Through the use of multiplicity of gene integration events as probes on the same genomic DNA blot, the integration event composition of each plant may be determined. Correlations may be established between the contributions of particular integration events to the phenotype of the plant. Only those plants that contain a desired combination of integration events may be advanced to maturity and used for pollination. DNA probes corresponding to particular transgene integration events are useful markers during the course of plant breeding to identify and combine particular integration events without having to grow the plants and assay the plants for agronomic performance.

It is expected that one or more restriction enzymes will be used to digest genomic DNA, either singly or in combinations. One of skill in the art will recognize that many different restriction enzymes will be useful and the choice of restriction enzyme will depend on the DNA sequence of the transgene integration event that is used as a probe and the DNA sequences in the genome surrounding the transgene. For a probe, one will want to use DNA or RNA sequences which will hybridize to the DNA used for transformation. One will select a restriction enzyme that produces a DNA fragment following hybridization that is identifiable as the transgene integration event. Thus, particularly useful restriction enzymes will be those which reveal polymorphisms that are genetically linked to specific transgenes or traits of interest.

### EXAMPLES

The following examples are offered by way of example, and are not intended to limit the scope of the invention in any manner.

### Example 1

### Pharmacological Inhibition of HSP90 in Arabidopsis Seedlings

The Arabidopsis genome contains seven closely related Hsp90 genes (Milioni and Hatzopoulos, 1997; Krishna and Gloor, 2001). Therefore, to reduce Hsp90 function, pharmacological inhibition was used. The drugs geldanamycin (GdA) and radicicol are structurally unrelated, but both make multiple, distinct contacts with residues in the unusual and evolutionary conserved nucleotide-binding pocket of Hsp90 and inhibit its function (Roe et al., 1999; Duta and Inouye, 2000). The drug-binding residues are poorly conserved in other proteins with this unconventional ATP-binding fold. An important advantage is that both drugs are inactivated by prolonged exposure to light, allowing the recovery and propagation of plants with early abnormal phenotypes.

In two ecotypes (accessions) examined, Columbia (Col) and Landsberg erecta (Ler), the drugs produced dosage-dependent phenotypes at concentrations that correlated with their affinities for Hsp90. For radicicol (K_{d} for binding Hsp90 19 nM), dosage-dependent phenotypes appeared between 2 nM and 50 nM; for GdA (K_{d} 1.2 μM) (Roe et al., 1999), phenotypes appeared between 0.15 μM and 5 1M. Above the K_{d}, plants exhibited multiple phenotypes and reduced viability. At lower concentrations, most plants remained healthy and unaffected while some exhibited strongly altered phenotypes. For example, at 1.0 μM GdA, 5-8% of seedlings showed strong morphological abnormalities, affecting shape, color, and expansion of cotyledons; shape, color and presence of true leaves; shape and length of hypocotyls; root morphology; and the orientation of rosettes, roots or whole seedlings. In most cases phenotypes were distinct and were not accompanied by a general failure to thrive. In the absence of the drug only 1-2% of plants showed variant morphologies, and these were much more subtle. Most importantly, the chemically unrelated GdA (a benzoquinone ansamycin) and radicicol (a macrolactone) produced the same spectrum of phenotypes.

Next, several Arabidopsis ecotypes were tested from diverse geographic locations and environments, including Cape Verde Island (Cvi), Shadara, Tsu-1, Mr-0, and Ts-1. Again, most plants bearing abnormal phenotypes on GdA had an otherwise healthy appearance. Particular phenotypes were not restricted to particular ecotypes, but the frequencies at which they appeared varied reproducibly between them. For example, in all ecotypes, rare plants exhibited abnormalities in true leaves including radially-symmetric leaves, deformed shapes, and missing leaves, but in Shadara ˜30% of seedlings had these phenotypes. Shadara also frequently produced distorted rosettes with juxtaposed cotyledons. Dwarfed plants with cotyledons that accumulated purple pigment were most common in Col. Curled hypocotyls were most frequent in Ler.

### Example 2

### Phenotypic Variation Specific to Recombinant Inbred Lines

The predisposition of different ecotypes to different phenotypes suggested a genetic contribution to the phenotypic variation buffered by Hsp90. To test this, recombinant inbred lines (RI lines) were examined, which originated from crosses between two ecotypes followed by single-seed self-propagation for eight generations. Each line should be homozygous at almost all loci, with different lines representing various mosaics of the parental genomes (Alonso-Blanco et al., 1998). If a decrease in Hsp90 function uncovers novel phenotypes due to natural variation, GdA-induced phenotypes should vary between RI lines but tend to be shared within them. Here, work with 50 RI lines derived from a cross between Cvi and Ler (Cvi/Ler, CS22477 base set, ABRC) is presented. These lines are characterized by high rates of recombination and low segregation distortion (Alonso-Blanco et al., 1998). Col/Ler RI lines behaved similarly, although phenotypic variation was not quite so dramatic.

The most striking IIsp90-buffered phenotypes in Cvi/Ler RI lines were distinct and restricted to particular lines (Table 1).

TABLE 1
RI line specific phenotypes on GdA and at 27° C.

| | | Frequency | | Frequency with | | Frequency | |
|---|---|---|---|---|---|---|---|
| | | without GdA at | | 1 μM GdA at | | without GdA at | |
| RI lines | RI line-specific phenotype | 22° C. | | 22° C. | | 27° C. | |
| 113 | Curled hypocotyl with root | 1^{a}/28^{b} | 3%^{c} | 9/28 | 32% | 9/28 | 32% |
| | partially extended into air | | | | | | |
| 082 | S-shaped rosette with | 2/112 | 2% | 64/201 | 32% | 53/56 | 95% |
| | vertically oriented leaf | | | | | | |
| | blade of cotyledons | | | | | | |
| 104 | Purple pigmentation of | 0/75 | | 29/84 | 34% | 0/47 | |
| | cotyledons and true leaves | | | | | | |
| 104 | Root morphology | 12/75 | 16% | 44/84 | 52% | 18/47 | 38% |
| 159 | Root morphology | 8/127 | 6% | 53/105 | 50% | 18/63 | 28% |
| 134 | Bent hypocotyls | 0/28 | | 49/56 | 88% | 19/41 | 46% |
| 122 | Distorted rosette, | 17/110 | 15% | 44/158 | 28% | 41/130 | 32% |
| | juxtaposed or fused | | | | | | |
| | cotyledons | | | | | | |
| 119 | Deformed, radially- | 2/28 | 7% | 19/28 | 68% | 25/28 | 90% |
| | symmetric and missing true | | | | | | |
| | leaves | | | | | | |
| 120 | Radially-symmetric and | 2/28 | 7% | 18/29 | 64% | 23/28 | 82% |
| | missing true leaves | | | | | | |

The most striking Hsp90-buffered phenotypes in Cvi/Ler RI lines were distinct and restricted to particular lines (Table 1). For example, seedlings of line CS22113 (113) developed normally for about eight days. Thereafter, their hypocotyls started to curl, lifting the root above the medium surface. By day 10, approximately 30% of the seedlings showed extreme hypocotyl curls and unusually large, round cotyledons. For seedlings of line 082, leaf blades of cotyledons were positioned vertically instead of horizontally, twisting the seedling rosette in an S-shape. Seedlings of lines 104 and 159 displayed a profuse increase in root hair growth. Some 104 seedlings also accumulated purple pigment in cotyledons and emerging adult leaves. For line 134 seedlings, arched hypocotyls were observed.

Other RI lines exhibited phenotypes that were variable but appeared related: fused cotyledons and distorted rosettes (122), radially-symmetric leaves and missing true leaves (119 and 120), with the addition of other leaf deformities (119). Both lines 119 and 120 also showed occasional trichomes on the abaxial side of the first true leaf.

Notably, a few seedlings in most lines exhibited altered phenotypes in the absence of GdA. Due to potential pitfalls of subjective analysis, all seedlings with detectable phenotypes were scored in Table 1. As with different ecotypes, these phenotypes were not only less frequent in the absence of GdA, but they were also generally much more subtle in character. They were, however, specific to individual lines and related to the generally stronger phenotypes observed with GdA. That is, while seedlings of line 113 sometimes exhibited slightly curled hypocotyls without GdA, none exhibited S-shaped rosettes, malformed true leaves, or abnormally abundant root-hair growth. Thus, GdA phenotypes were based on uncovering a genetic predisposition of RI lines to certain traits (Table 1), rather than to irrelevant drug effects. Moreover, some of the traits (e.g. juxtaposed cotyledons) observed in RI lines were never observed in their parents. In a specific embodiment, the enrichment of RI lines for different combinations of their parental genomes produces the potential for a wide variety of genetically determined traits, and these are strongly expressed when Hsp90 chaperone capacity falls below a certain threshold. (Partial penetrance of strong phenotypes in RI line populations is discussed below).

The diversity of the phenotypes observed indicates that the level of Hsp90 inhibition required to uncover them is relatively modest; it must not interfere with developmental pathways that have acquired an absolute dependence on Hsp90 in this species. Moreover, the robust health of most seedlings displaying strong phenotypes demonstrates that the levels of inhibition employed are not severe enough to interfere with any general housekeeping functions of Hsp90. However, because Hsp90 contributes to heat-shock protein (Hsp) regulation through its interaction with heat-shock transcription factors, the conditions might induce a general heat-shock response. To test this, eight randomly selected lines, including two that produced the strongest phenotypes (113, 119), were examined for expression of Hsp101 and Hsp70 by Western blotting. All exhibited normal Hsp inductions with a brief 38° C. heat shock (Queitsch et al., 2000). None showed detectable Hsp expression at normal temperature (22° C.) in the presence of GdA. Certainly, some phenotypes are likely to be influenced by other Hsps. However, the threshold of Hsp90 inhibition required to uncover the specific polymorphisms that can give rise to strong phenotypes was generally lower than that required to produce a detectable stress response.

### Example 3

### Phenotypes Uncovered by Environmental Change

To determine if Hsp90-buffered variation is subject to exposure by natural environmental change, Cvi/Ler RI line seedlings were grown in the absence of GdA at 27° C. (As with GdA, this moderate temperature increase did not induce a detectable Hsp induction). All but one of the lines that exhibited strong phenotypes with GdA exhibited the same phenotypes at 27° C. Even in the exceptional case, line 104, one of its two GdA-induced phenotypes (abundant root hair growth) was expressed. The ability of environmental change to reveal previously hidden genetic variation was precise: in the other 42 lines examined, some seedlings exhibited other phenotypes at 27° C., but rarely these.

Other environmental conditions also had the capacity to uncover Hsp90 buffered traits. In the presence of GdA the roots of RI lines 104 and 159 exhibited profuse root-hair growth (Table 1) and were often located at the agar surface. These lines were also the most likely to produce a phenotype in the absence of GdA. To determine if root phenotypes in lines 104 and 159 might be influenced by the nature of the growth substratum, they were grown on medium containing phytagel (agar-like medium) at different concentrations (2.0, 2.4, 3.0, and 4.0 g/L). In every case root morphology phenotypes were stronger with GdA than without GdA. However, in the absence of GdA, the root hair growth of 104 seedlings became more profuse as phytagel concentration increased. In the extreme case (4.0 g/L), root hairs were much more abundant than normal, albeit not as profuse as at low phytagel concentrations with GdA. Seedlings of parental ecotypes Cvi and Ler and of the other 48 RI lines tested did not respond with root hair profusion to increased phytagel concentrations, to GdA, or to growth at 27° C. Clearly, the ability to increase root-hair growth is an Hsp90-buffered trait, whose expression depends both upon the genetic background and its interaction with the environment.

### Example 4

### HSP90-Dependent Phenotypic Plasticity

Next, developmental plasticity was tested for being broadly dependent on Hsp90 function. The dark response is a classic example of developmental plasticity: in contrast to light-grown Arabidopsis seedlings, dark-grown seedlings have elongated hypocotyls; small, yellow, and non-expanded cotyledons; and short roots. Cvi and Ler ecotypes differ in hypocotyl length both in the light and dark. RI lines and their parents are sorted by the degree of hypocotyl elongation they exhibited in the dark in the absence of GdA. Notably, some differences between RI line responses were greater than the difference between the responses of the parental lines (transgression). This indicates that multiple genes affecting elongation were contributed by Cvi and Ler parents and had segregated broadly in the original cross.

GdA effects were uniform within lines but differed dramatically between lines. In some (e.g. 077 and 129), the drug had very little effect. In others (e.g. 086 and 116), it reduced hypocotyl growth to nearly the values obtained in white light. GdA did not simply intensify the pattern of variation observed without the drug. Lines with naturally long or with naturally short hypocotyls were found both in the group that exhibited little change with Hsp90 inhibition and in the group that showed strong effects. Both parental lines showed moderate reductions in hypocotyl length. Thus, the degree to which the dark response was affected by modest changes in Hsp90 function segregated in RI lines, producing lines with extreme Hsp90 dependence and others with much less.

Several other traits related to developmental plasticity were examined: 1) root growth in the dark promoted by sucrose in the medium, 2) germination in the dark with or without a light pulse, 3) the ability of cotyledons to green after seven days in the dark followed by two days in the light, and 4) the ability of roots to respond to a change in the direction of gravity.

If Hsp90 buffers polymorphisms in many different environmental response pathways, Applicants would expect that the Hsp90-dependence of different traits varies both within a given RI line and between RI lines. Indeed, in the same seedlings, measured on the same day, GdA affected different traits in different ways. For example, consider lines 132 and 079 (blue font): hypocotyl elongation and greening were more strongly affected in 132; root elongation and germination were more affected in 079. Gravitropism responses were assessed in a separate experiment by rotating plants grown on vertical plates by 90°. This trait also showed different sensitivities to GdA in different lines, including one (129) in which roots turned more efficiently with GdA than without (Wilcoxon two-sample test, p£0.018). Most notably, GdA's effects on specific plasticity responses appeared largely independent of each other (Spearman's rank tests, turning versus root elongation, p£0.7; versus hypocotyl elongation, p£0.3; versus germination rate, p£0.3; germination rate versus root elongation, p£0.6; versus hypocotyl elongation, p£0.5).

Thus, the Hsp90-dependent variation was due neither to random nor to line-specific differences in drug-uptake or toxicity. Rather, it resulted from genetic variation in different environmental response pathways that rendered them more or less dependent on full Hsp90 function. By QTL mapping, the uncovered phenotypic variation in hypocotyl elongation did not map to the cytoplasmic Hsp90 gene cluster. This confirms that, for this trait, the phenotypic variation uncovered by Hsp90 inhibition is not due to polymorphisms in Hsp90 itself, but to a wide variety of polymorphisms elsewhere in the genome.

Thus, three conclusions follow: Hsp90 plays an important role in many aspects of developmental plasticity; these roles vary in different genetic backgrounds; and the dependencies of different pathways on Hsp90 segregate independently of each other.

### Example 5

### HSP90 Buffers Developmental Stability in Crosses Between Diverse Genomes

Given the near homozygosity of inbred lines and ecotypes, the partial penetrance observed for certain phenotypes is surprising. That is, although some morphogenetic traits were highly penetrant (e.g. root development in line 104 with GdA at 4.0 g/L phytagel and S-shaped rosettes in line 082 at 27° C.), most were only partially penetrant. Similarly, for some developmentally plastic traits (e.g. germination, gravitropism), the responses of individual seedlings varied within a line. One possible explanation for this phenotypic variability is the segregation of genetic variation. Low levels of heterozygosity are present in RI lines (˜1 site/5000 bp) and might also be present in ecotypes. Indeed, this was demonstrably true in at least one case tested. Up to 30% of Shadara seedlings exhibited deformed, missing, or radially-symmetric true leaves in the presence of GdA. Even without the drug, subtle abnormalities of true leaf development appeared far more frequently (˜3%-6%) than in other ecotypes (less than 1%). Plants that exhibited such subtle abnormalities in the absence of GdA were selfed. Phenotype frequency increased dramatically in the progeny (50% exhibited the phenotype without GdA, 75% with GdA). Thus, genes contributing to abnormal leaf development are still segregating in the Shadara seeds that were sampled and can be enriched by selective breeding to produce increased phenotypic penetrance. In contrast, in RI lines displaying partial penetrance of an altered phenotype (e.g. line CS1941, radially-symmetric and missing true leaves), selfing and crossing the affected progeny did not increase penetrance in the next generation. In these cases, partial penetrance is not due to segregating genetic variation but is more likely due to stochastic processes that influence the propensity of a given genome to produce a particular trait.

To investigate the interplay of genetic variation, stochastic processes, and their dependence on the Hsp90 buffering system, Applicants tested crosses of different, nearly homozygous accessions (Col x Ler and Ler x Cvi) that are believed to have been recentlyevolving independently. If stochastic events contribute to phenotypes predisposed by genotype, then mixing genomes might disrupt developmental stability in F1 progeny. Further, if Hsp90 buffers developmental stability against such events, reducing Hsp90 function in nearly identical F1 progeny should increase phenotypic variance. Indeed, F1 progeny exhibited higher frequencies of subtle altered phenotypes than their parental accessions even without GdA (frequency 5-7%). In the presence of GdA, not only did the frequency of abnormal phenotypes increase dramatically (up to 25%), but also the severity and complexity of the phenotypes increased. Phenotypes were stronger and more frequent in seedlings with a maternal contribution from Ler (˜20 to 25% affected seedlings) than with maternal contributions from Col or Cvi (˜10 to 15%).

Notably, the developmental diversity of F₁ seedlings in the presence of GdA was not due to reduced viability. Indeed, when the concentration of GdA was doubled (to 2 μM), Col/Ler F₁ seedlings exhibited an even greater abundance of phenotypes, but the majority of these seedlings were more vigorous and advanced in development than those of either parental ecotype at the same GdA concentration. When buffering functions were challenged by a higher growth temperature (27° C.) instead of by GdA, Col/Ler-F₁ seedlings exhibited a similarly enhanced diversity of phenotypes compared to parental ecotypes. Again, F₁ seedlings were generally larger and healthier than seedlings of the parental ecotypes. Preliminary analysis of other crosses suggests that the effects of heterozygosity on developmental stability and stress tolerance may vary, depending on the genetic divergence of the ecotypes and the nature of the stress.

### Example 6

### Significance of the Present Invention

The work described herein establishes three aspects of Hsp90 function in organismal biology. First, its role in buffering the expression of genetic variation (Rutherford and Lindquist, 1998) is conserved across plant and animal kingdoms. Although it is difficult to predict what might prove adaptive in evolution, it is noteworthy that the phenotypes revealed in plants by challenging Hsp90 buffering capacity are not “monstrous” in character (as they might be described in fruit flies). Indeed, some would seem plausibly advantageous under particular conditions, e.g. altered leaf shapes, purple pigment accumulation, different degrees of hypocotyl extension, and germination in the dark. Most notably, the majority of Hsp90-buffered traits tested were also uncovered by moderate changes in growth conditions (i.e., a mild temperature increase or an increase in the density of the substratum). This establishes that the Hsp90 buffer has global effects on the storage of cryptic polymorphisms and their release in response to shifting environments, converting neutral to non-neutral variation.

Second, Hsp90 profoundly affects developmental plasticity. Plasticity allows morphogenetic variation in response to circumstances and environments and is particularly salient in plants. The observations provided herein indicate that Hsp90 functions at the interface between genotypes and environments, a unique vantage point from which to influence the dynamic nature of developmental processes. Most remarkably, the degree to which the plasticity of individual traits depends upon Hsp90 varied greatly between RI lines, and different traits exhibited different levels of Hsp90 dependence within each RI line. By QTL mapping, this variation does not map to the cytoplasmic Hsp90 gene cluster. Apparently, the genetic networks that affect both the likelihood that a particular trait might appear, as well as the extent to which it may respond to changes in the environment, are commonly polymorphic with respect to the influence of Hsp90. In a specific embodiment, in the face of changing selective pressures, Hsp90 buffering provides an avenue by which populations can evolve different genotypic states-from those that produce a particular trait, to those in which the trait dynamically responds to the environment, to those in which a different developmental endpoint has become a fixed characteristic.

Third, Hsp90 buffers developmental stability against stochastic processes. Individual seedlings in nearly isogenic RI lines and accessions produced strong phenotypes when Hsp90 function was reduced, but these were only partially penetrant. Also, the nearly genetically identical F₁ progeny of crosses between Col and Ler parents produced an unprecedented diversity of phenotypes when Hsp90 function was challenged. These phenotypes were not due to a general loss of vigor, as F₁ progeny from the same cross were generally more robust than either parent under conditions of increased stress. Thus, Hsp90 normally acts to reduce the likelihood that stochastic events will alter the deterministic unfolding of a multitude of developmental programs.

These seemingly diverse effects of Hsp90 are readily encompassed within a single, simple molecular framework. Hsp90's biochemical function is to chaperone metastable proteins, including diverse regulators of growth and development. In doing so, it stabilizes conformations of proteins that would otherwise be prone to misfolding and potentiates the proteins' capacity to be activated in the proper time and place, through associations with partner proteins, ligand binding, post-translational modifications, and correct localization (Buchner, 1999; Mayer and Bukau, 1999; Young et al., 2001). Under stressful conditions Hsp90 is induced as part of a general response to the strains on protein folding. Polymorphisms with the potential to alter a particular trait may be so enriched in individual genomes that the pathway exceeds Hsp90's ability to maintain stability. Indeed, even in the absence of stress, some populations may be so close to this threshold that stochastic events in development will produce a few individuals expressing the altered trait. Once the pathway is diverted, the expression of a new trait may become robust through the influence of auto-regulatory feedback loops, self-perpetuating protein conformations, and developmental windows. Thus, Hsp90 contributes to phenotypic variance by buffering the functional state (folding, accumulation, local concentration) of gene products that contribute to altered traits through the effects of chance, genotypes, and environments.

From a population geneticist viewpoint, buffering systems like Hsp90 may decrease selection on nucleotide substitutions, allowing storage of an expanded spectrum of selectively nearly neutral ones. Under stable environmental conditions, a population arrives at a local fitness optimum in an adaptive landscape. Given that natural selection can only further increase the fitness of a population, it is a perplexing evolutionary question how a population might move to a different local optimum without an intervening period of reduced fitness (adaptive valley). Previously proposed mechanisms include genetic drift in small populations, compensatory mutations, and gene conversion (Kumura, 1991; Ludwig et al., 2000; Hansen et al., 2000). As a byproduct of its biochemical function, Hsp90 may allow the neutral accumulation of potentially selectable polymorphisms, thereby providing a molecular means by which adaptive peak shifts in large populations may occur without passing through an adaptive valley. None of the other mechanisms discussed can be modulated by environmental contingencies.

Finally, deliberate manipulation of the Hsp90 buffer may offer an opportunity to speed the identification of genetic variation for academic investigations and commercial applications. In some cases, developmental pathways might be deciphered more rapidly by taking advantage of multiple, subtle polymorphisms contributing to a trait than by traditional approaches of sequentially identifying strong alleles, enhancers, and suppressors. Harnessing such variation to produce valuable phenotypes would also bypass public concern about the application of transgenic methods. The results indicate possible connections to medical maladies with stochastic genotypic and environmental etiology, as they provide a global molecular explanation for why genotype does not always directly translate into phenotype and locate Hsp90 at the intersection of genotype, environment, and development.

### Example 7

### Materials and Methods

The following represents exemplary materials and methods for the present invention. However, a skilled artisan is aware of alternative means to achieve the same or similar goals.

Plant Material and Growth Conditions

Ecotype seeds (Mr-0, Shadara, Ts-1, Tsu-1) and RI lines (CS22477, CS 1899) were received from the Arabidopsis Biological Resource Center (ABRC). Columbia (Col-0), Ler, and Cvi seeds were laboratory stocks propagated from seeds received from D. Preuss (University of Chicago, Ill.).

Ecotypes were crossed several times in independent experiments to produce F₁ seeds. To minimize variation in growth conditions, in all experiments surface-sterilized seeds were plated with equal spacing on germination (GM) medium (per liter: 1× Murashige and Skoog medium (Sigma), 1.0 mL Murashige and Skoog vitamins (Sigma), 10 g sucrose, pH 5.7 with KOH, 2.4 g Phytagel (Sigma), unless otherwise indicated) containing either 1 μM GdA (from 1 mg/mL stock in DMSO) or equivalent concentration of DMSO alone (0.056%). Plates were wrapped in foil to allow germination while avoiding degradation of light-sensitive GdA and incubated at 22° C. after cold treatment for 24-48 hrs. Plates were unwrapped after 48 hrs at 22° C., were incubated in continuous light at ˜150 μmol m⁻² sec⁻¹ (unless otherwise indicated), and were analyzed after eight and ten days of growth.

Experiments in Altered Environments

Incubation at 27° C. increased growth rates (by ˜0.5-1 day after 8 days), and GdA decreased growth rates (by ˜0.5-1 day) relative to plants grown without GdA at 22° C. To accommodate these differences, one set of seeds (˜28 per RI line) was plated with GdA on day one; a second set was plated without GdA for growth at 22° C. on day two. On day three, three sets were plated: one for growth at 27° C. and two for growth at 22° C., with and without GdA. To minimize variation and provide more objective comparisons, two RI lines were plated together on each square plate. All sets were cold treated for 48 hrs. Foil-wrapped plates incubated at 22° C. were treated as described above. Since development was more rapid at 27° C., these plates were unwrapped after 24 hrs instead of 48 hrs. Phenotypes were scored at eight and ten days.

For growth at increased phytagel concentrations, parental ecotypes and RI lines 104 and 159 were grown together on square plates and were analyzed on day ten and fourteen.

Developmental Plasticity Responses

Seedlings can respond to subtle environmental changes that are very difficult to control (e.g. small variations in incubator conditions, slight changes in substratum). Values for individual lines varied somewhat between experiments, but within experiments seedlings on multiple replicate plates behaved similarly. For example, for the dark response of hypocotyls and roots, fifty seeds for each RI line were plated on vertical plates (ten seeds per plate) and intermixed with multiple RI lines in the incubator. A second screen for the dark response analyzed ten seeds for each RI line with the addition of a 2 hr light pulse and 48 hrs cold treatment instead of 24 hrs. Relative hypocotyl lengths were similar to those in the other experiment for most but not all RI lines (the hypocotyls of lines 117 and 086 were much longer than those of light-grown seedlings).

After incubation for seven days in the dark, hypocotyl and root lengths were measured on digitalized images using Scion Image. The number of germinated seeds for each line, morphological characteristics of seedlings grown with and without GdA, and greening after subsequent light exposure for 48 hrs were documented.

The rate of GdA decay in the light was assessed phenotypically. GdA-containing plates were pre-exposed to light for various periods before seeds were plated, and hypocotyl growth was measured after 7 days in the dark as before. Light exposures of 2-4 hrs had little effect on GdA potency; 48 hrs eliminated it.

Because GdA can affect root extension, the response to a change in the direction of gravity was tested at GdA concentrations of 0.75 μM and 1 μM to ensure that a set of seedlings was available for each RI line with root growth comparable to controls. Forty seeds for each condition were plated, cold treated for 48 hrs, and incubated at 22° C. in the dark for four days. Root growth and germination were scored, and plates were then turned by 90°.

One skilled in the art readily appreciates that the patent invention is well adapted to carry out the objectives and obtain the ends and advantages mentioned as well as those inherent therein. Methods, compositions, plants, sequences, plasmids, vectors, treatments, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

### PATENTS

PCT Application WO 9217598

PCT Application WO 94/09699

PCT Application WO 95/06128

PCT Application WO 95/06128

U.S. Pat. No. 5,302,523

U.S. Pat. No. 5,322,783

U.S. Pat. No. 5,384,253

U.S. Pat. No. 5,384,253

U.S. Pat. No. 5,384,253

U.S. Pat. No. 5,464,765

U.S. Pat. No. 5,508,184

U.S. Pat. No. 5,538,877

U.S. Pat. No. 5,538,880

U.S. Pat. No. 5,538,880

U.S. Pat. No. 5,550,318

U.S. Pat. No. 5,550,318

U.S. Pat. No. 5,563,055

U.S. Pat. No. 5,563,055

U.S. Pat. No. 5,563,055

U.S. Pat. No. 5,563,055

U.S. Pat. No. 5,563,055

U.S. Pat. No. 5,591,616

U.S. Pat. No. 5,591,616

U.S. Pat. No. 5,610,042

### PUBLICATIONS

Abbott, R. J. & Gomes, M. F. Heredity 62, 411-418 (1989).

Abdullah et al., Biotechnology, 4:1087, 1986.

Abel et al., Science, 232:738-743, 1986.

Alonso-Blanco, C. et al. Plant J 14, 259-271 (1998).

Araki et al., J. Mol. Biol. 225(1):25-37, 1992.

Armaleo et al., Curr. Genet. 17(2):97-103, 1990.

Armstrong et al., Maize Genetics Coop Newsletter, 65:92-93, 1991.

Bansal et al., Proc. Nat'l Acad. Sci. USA, 89:3654-3658, 1992.

Barkai-Golan et al., Arch. Microbiol., 116:119-124, 1978.

Bates, “Genetic transformation of plants by protoplast electroporation,” Mol Biotechnol., 2(2):135-145, 1994.

Battraw and Hall, “Stable transformation of sorghum-bicolor protoplasts with chimeric neomycin phosphotransferase II and beta glucuronidase genes,” Theor. App. Genet., 82(2):161-168, 1991.

Becker et al., “Fertile transgenic wheat from microprojectile bombardment of scutellar tissue,” Plant J., 5(2):299-307, 1994.

Belanger and Kriz, Genet., 129:863-872, 1991.

Bellus, J. Macromol. Sci. Pure Appl. Chem., 1(1):1355-1376, 1994.

Bernal-Lugo and Leopold, Plant Physiol., 98:1207-1210, 1992.

Berzal-Herranz et al., Genes and Devel., 6:129-134, 1992.

Bevan et al., Nucleic Acids Research, 11(2):369-385, 1983.

Bhattacharjee; An; Gupta, J. Plant Bioch. and Biotech. 6, (2):69-73. 1997.

Blackman et al., Plant Physiol., 100:225-230, 1992.

Bol et al., Annu. Rev. Phytopath., 28:113-138, 1990.

Bouchez et al., EMBO Journal, 8(13):4197-4204, 1989.

Bower et al., The Plant Journal, 2:409-416. 1992.

Bowler et al., Ann Rev. Plant Physiol., 43:83-116, 1992.

Branson and Guss, Proceedings North Central Branch Entomological Society of America, 27:91-95, 1972.

Broakaert et al., Science, 245:1100-1102, 1989.

Buchanan-Wollaston et al., Plant Cell Reports 11:627-631. 1992.

Buchner, J. Trends Biochem. Sci. 24, 136-141 (1999).

Buising and Benbow, Mol. Gen. Genet., 243:71-81, 1994.

Callahan, H. S., Pigliucci, M. & Schlichting, C. D. Bioessays 19, 519-525 (1997).

Callis et al., Genes Dev., 1:1183-1200, 1987.

Callis, Fromm, Walbot, Genes Dev., 1:1183-1200, 1987.

Campbell (ed.), In: Avermectin and Abamectin, 1989.

Capaldi et al., Biochem. Biophys. Res. Comm., 76:425, 1977.

Casa et al., Proc. Nat'l Acad. Sci. USA, 90(23):11212-11216, 1993.

Casas et al., “Transgenic sorghum plants via microprojectile bombardment,” Proc. Natl. Acad. Sci. USA, 90(23):11212-11216, 1993.

Cashmore et al., Gen. Eng. of Plants, Plenum Press, New York, 29-38, 1983.

Cech et al., Cell, 27:487-496, 1981.

Chandler et al., The Plant Cell, 1:1175-1183, 1989.

Chau et al., Science, 244:174-181, 1989.

Chomet et al., EMBO J., 6:295-302, 1987.

Chowrira et al, J. Biol. Chem., 269:25856-25864, 1994.

Christou; Murphy; Swain, Proc. Nat'l Acad. Sci. U S A, 84(12):3962-3966, 1987.

Chu et al., Scientia Sinica, 18:659-668, 1975.

Coe et al., In: Corn and Corn Improvement, 81-258, 1988.

Conkling et al., Plant Physiol., 93:1203-1211, 1990.

Cordero, Raventos, San Segundo, Plant J., 6(2)141-150, 1994.

Coxson et al., Biotropica, 24:121-133, 1992.

Cretin and Puigdomenech, Plant Mol. Biol. 15(5):783-785, 1990

Cuozzo et al., Bio/Technology, 6:549-553, 1988.

Cutler et al, J. Plant Physiol., 135:351-354, 1989.

Czapla and Lang, J. Econ. Entomol., 83:2480-2485, 1990.

Davies et al., Plant Physiol., 93:588-595, 1990.

De Block et al., The EMBO Journal, 6(9):2513-2518, 1987.

De Block, De Brouwer, Tenning, Plant Physiol., 91:694-701, 1989.

Dellaporta et al., In: Chromosome Structure and Function: Impact of New Concepts, 18th Stadler Genetics Symposium, 11:263-282, 1988.

Deng, X. W. Symp. Soc. Exp. Biol. 51, 93-96 (1998).

Dennis et al., Nucl. Acids Res., 12(9):3983-4000, 1984.

Depicker et al., Plant Cell Reports, 7:63-66, 1988.

D'Halluin et al., “Transgenic maize plants by tissue electroporation,” Plant Cell, 4(12):1495-1505, 1992.

D'Halluin et al., Plant Cell, 4(12):1495-1505, 1992.

Didierjean et al., Plant Mol Biol 18(4):847-849, 1992.

Dure et al., Plant Molecular Biology, 12:475-486, 1989.

Durrant, A. Heredity 17, 27-61 (1962).

Dutta, R. & Inouye, M. Trends Biochem. Sci. 25, 24-28 (2000).

Ebert et al., 84:5745-5749, Proc. Nat'l Acad. Sci. USA, 1987

Ellis et al., EMBO Journal, 6(11):3203-3208, 1987.

Enomoto, et al., J. Bacteriol., 6(2):663-668, 1983.

Erdmann et al., Mol. Jour. Gen. Micro., 138:363-368, 1992.

Feinberg and Vogelstein, Anal. Biochem., 132:6-13, 1983.

Finkle et al., Plant Sci., 42:133-140, 1985.

Fitzpatrick, Gen. Engineering News, 22:7, 1993.

Forster and Symons, Cell, 49:211-220, 1987.

Fraley et al., Bio/Technology, 3:629-635, 1985.

Franken et al., EMBO J., 10(9):2605-2612, 1991.

Fransz, de Ruijter, Schel, Plant Cell Reports, 8:67-70, 1989.

Fromm et al., “_Stable transformation of maize after gene transfer by electroporation,” Nature, 319:791-793, 1986

Gallie et al., The Plant Cell, 1:301-311, 1989.

Gatehouse et al., J. Sci. Food. Agric., 35:373-380, 1984.

Gelvin et al., In: Plant Molecular Biology Manual, 1990.

Gerlach et al, Nature 328:802-805, 1987.

Ghosh-Biswas et al., “Transgenic Indica rice (Oryza sativa L.) plants obtained by direct gene transfer to protoplasts,” J. Biotechnol., 32(1):1-10, 1994.

Goddemeier et al., Plant Mol. Bio. 36:799-802, 1998.

Golic and Lindquist, Cell, 59:3, 499-509. 1989.

Gomez et al., Nature, 334:262-263, 1988.

Goring et al., Proc. Nat'l Acad. Sci. USA, 88:1770-1774, 1991.

Guerrero et al., Plant Molecular Biology, 15:11-26, 1990.

Gupta et al., Proc. Nat'l Acad. Sci. USA, 90:1629-1633, 1993.

Hagio et al., “Stable transformation of sorghum cell cultures after bombardment with DNA coated microprojectiles,” Plant Cell Rep., 10(5):260-264, 1991.

Hamilton et al., Proc. Nat'l Acad. Sci. USA, 93(18):9975-9979, 1996.

Hammock et al., Nature, 344:458-461, 1990.

Hansen, T. F., Carter, A. J. & Chiu, C. H. Gene conversion may aid adaptive peak shifts. J. Theor. Biol. 207, 495-511 (2001).

Haseloff and Gerlach, Nature, 334:585-591, 1988.

Haseloff et al., Proc. Nat'l Acad. Sci. USA 94(6):2122-2127, 1997.

He et al., Plant Cell Reports, 14 (2-3):192-196, 1994.

Hemenway et al., The EMBO J., 7:1273-1280, 1988.

Hensgens et al., “Transient and stable expression of gusA fusions with rice genes in rice, barley and perennial ryegrass,” Plant Mol. Biol., 22(6):1101-1127, 1993.

Hiei et al., “Transformation of rice mediated by agrobacterium tumefaciens,” Plant Mol. Biol., 35(1-2):205-218, 1997.

Hilder et al., Nature, 330:160-163, 1987.

Hinchee et al., Bio/technol., 6:915-922, 1988.

Hou and Lin, Plant Physiology, 111: 166, 1996.

Hudspeth and Grula, Plant Mol. Biol., 12:579-589, 1989.

Ikeda et al., J. Bacteriol., 169:5615-5621, 1987.

Ikuta et al., Bio/technol., 8:241-242, 1990.

Ishida et al., “High efficiency transformation of maize (Zea mays L.) mediated by Agrobacterium tumefaciens,” Nat Biotechnol., 14:745-50, 1996.

Jefferson R. A., Plant Mol. Biol. Rep., 5:387-405, 1987.

Johnson et al., Proc. Nat'l Acad. Sci. USA, 86:9871-9875, 1989.

Joshi, Nucleic Acids Res., 15:6643-6653, 1987.

Joyce, Nature, 338:217-244, 1989.

Kaasen et al., J. Bacteriology, 174:889-898, 1992.

Kaeppler et al., “Silicon carbide fiber-mediated stable transformation of plant cells,” Theor. Appl. Genet., 84(5-6):560-566, 1992.

Kaeppler et al., Plant Cell Reports 9: 415-418, 1990.

Kaeppler, Somers, Rines, Cockburn, Theor. Appl. Genet., 84(5-6):560-566, 1992.

Karsten et al., Botanica Marina, 35:11-19, 1992.

Katz et al., J. Gen. Microbiol., 129:2703-2714, 1983.

Keller et al., EMBO J., 8(5):1309-1314, 1989.

Kim and Cech, Proc. Nat'l Acad. Sci. USA, 84:8788-8792, 1987.

Kimura, M. Proc. Natl. Acad. Sci. USA 88, 5969-5973 (1991).

Klee et al., Bio-Technology, 3(7):637-642, 1985.

Knittel et al., Plant Cell Reports, 14(2-3):81-86, 1994.

Kohler et al., Plant Mol. Biol., 29(6):1293-1298, 1995.

Koster and Leopold, Plant Physiol., 88:829-832, 1988.

Krishna, P. & Gloor, G. Cell Stress Chaperones 6, 238-246 (2001).

Kriz, Boston, Larkins, Mol. Gen. Genet., 207(1):90-98, 1987.

Kuittinen, H., Mattila, A. & Savolainen, O. Heredity 79, 144-152 (1997).

Kunkel et al., Methods Enzymol, 154:367-382, 1987.

Langridge and Feix, Cell, 34:1015-1022, 1983.

Langridge et al., Proc. Nat'l Acad. Sci. USA, 86:3219-3223, 1989.

Laufs et al., Proc. Nat'l Acad. Sci., 7752-7756, 1990.

Lawton et al., Plant Mol. Biol. 9:315-324, 1987.

Lazzeri, “Stable transformation of barley via direct DNA uptake. Electroporation- and PEG-mediated protoplast transformation,” Methods Mol. Biol., 49:95-106, 1995.

Lee and Saier, J. of Bacteriol., 153-685, 1983.

Lee et al., Korean J. Genet., 11(2):65-72, 1989.

Levings, Science, 250:942-947, 1990.

Lieber and Strauss, Mol. Cell. Biol., 15: 540-551, 1995.

Lindstrom et al., Developmental Genetics, 11: 160, 1990.

Loomis et al., J. Expt. Zoology, 252:9-15, 1989.

Lorz et al., Mol Gen Genet, 199:178-182, 1985.

Ludwig, M. Z., Bergman, C., Patel, N.H. & Kreitman, M. Nature 403, 564-567 (2000).

Ma et al., Nature, 334:631-633, 1988.

Maas et al., Methods Cell Biol., 50:383-399, 1995.

Maeser et al, Mol. Gen. Genet., 230(1-2):170-176, 1991.

Mar Alba, M. et al. 1994. 6(6). 825-834.

Marcotte et al., Nature, 335:454, 1988.

Mariani et al., Nature, 347:737-741, 1990.

Martinez, Martin, Cerff, J. Mol. Biol., 208(4):551-565, 1989.

Mather, K. Heredity 7, 297-336 (1953).

Mayer, M. P. & Bukau, B. Curr. Biol. 9, R322-R325 (1999).

McCabe, Martinell, Bio-Technology, 11 (5):596-598, 1993.

McCormac et al., Euphytica, v. 99 (1) p. 17-25 :. 1998.

McElroy et al., Mol. Gen. Genet., 231:150-160, 1991.

McElroy, Zhang, Cao, Wu, Plant Cell, 2:163-171, 1990.

McLaren, A. Trends Genet. 15, 169-171 (1999).

Meagher, Int. Rev. Cytol., 125:139-163, 1991.

Michel and Westhof, J. Mol. Biol., 216:585-610, 1990.

Milioni, D. & Hatzopoulos, P. Plant MoL Biol. 35, 955-961 (1997).

Mundy and Chua, The EMBO J., 7:2279-2286, 1988.

Murakami et al., Mol. Gen. Genet., 205:42-50, 1986.

Murashige and Skoog, Physiol. Plant., 15:473-497, 1962.

Murdock et al., Phytochemistry, 29:85-89, 1990.

Murray et al., Nucleic Acids Research, 17:(2)477-498, 1989.

Nagatani et al., “DNA delivery into rice cells and transformation using silicon carbide whiskers,” Biotech. Tech., 11(7):471-473, 1997.

Napoli, Lemieux, Jorgensen, Plant Cell, 2:279-289, 1990.

Odell et al., Nature, 313:810-812, 1985.

Ogawa et al., Sci. Rep., 13:42-48, 1973.

Omirulleh et al., “Activity of a chimeric promoter with the doubled CaMV 35S enhancer element in protoplast-derived cells and transgenic plants in maize.” Plant Mol. Biol., 21:415-28, 1993.

Ow et al., Science, 234:856-859, 1986.

Palukaitis et al., Virology, 99:145-151, 1979.

Perlak et al., Proc. Nat'l Acad. Sci. USA, 88:3324-3328, 1991.

Perriman et al., Gene, 113:157-163, 1992.

Phi-Van et al., Mol. Cell. Biol., 10:2302-2307, 1990.

Piatkowski et al., Plant Physiol., 94:1682-1688, 1990.

Pignon et al., Hum. Mutat., 3:126-132, 1994.

Poszkowski et al., EMBO J., 3:2719, 1989.

Potrykus et al., Mol. Gen. Genet., 199:183-188, 1985.

Poulsen et al., Mol. Gen. Genet., 205(2):193-200, 1986.

Prasher et al., Biochem. Biophys. Res. Commun., 126(3):1259-1268, 1985.

Prody et al., Science, 231:1577-1580, 1986.

Queitsch, C., Hong, S. W., Vierling, E. & Lindquist, S. Plant Cell 12, 479-492 (2000).

Quigley, Brinkman, Martin, Cerff, J. Mol. Evol., 29(5):412-421, 1989.

Ralston, English, Dooner, Genet., 119(1): 185-197, 1988.

Reece, “The actin gene family of rice (Oryza sativa L),” Ph.D. thesis, Cornell University, Ithaca, N.Y., 1988.

Reece, McElroy, Wu, Plant Mol. Biol., 14:621-624, 1990.

Reed et al., J. Gen. Microbiology, 130:1-4, 1984.

Reichel et al., Proc. Nat'l Acad. Sci. USA, 93 (12) p. 5888-5893. 1996

Reina et al., Nucl. Acids Res., 18(21):6426, 1990.

Reinhold-Hurek and Shub, Nature, 357:173-176, 1992.

Rensburg et al., J. Plant Physiol., 141:188-194, 1993.

Rhodes et al., “Transformation of maize by electroporation of embryos,” Methods Mol. Biol., 55:121-131, 1995.

Ritala et al., “Fertile transgenic barley to particle bombardment of immature embryos,” Plant Mol. Biol., 24(2):317-325, 1994.

Rochester, Winer, Shah, EMBO J., 5:451-458, 1986.

Roe, S. M. et al. J. Med. Chem. 42, 260-266 (1999).

Rogers et al., Methods Enzymol., 153:253-277, 1987.

Rutherford, S. L. & Lindquist, S. Nature 396, 336-342 (1998).

Sambrook, Fritsch, and Maniatis, In Molecular Cloning: A Laboratory Manual, Second edition, Cold Spring Harbor Laboratory, Cold Spring Harbor, N.Y., 1989.

Sauer, Mol. and Cell. Biol., 7: 2087-2096. 1987.

Schwob et al., Plant J 4(3):423-432, 1993.

Shagan and Bar-Zvi, Plant Physiol., 101:1397-1398, 1993.

Shapiro, In: Mobile Genetic Elements, 1983.

Sheen et al., Plant Journal, 8(5):777-784, 1995.

Shimoni et al., “A recombinant protein of two high molelcular weight glutenins alters gluten polymer formation in transgenic wheat,” J. Biol. Chem., 272:15488-15495, 1997.

Shure et al., Cell, 35:225-233, 1983.

Simpson, Filipowicz, Plant Mol. Bio., 32:1-41, 1996.

Simpson, Science, 233:34, 1986.

Singsit et al., “Expression of a Bacillus thuringiensis cryIA(c) gene in transgenic peanut plants and its efficacy against lesser cornstalk borer,” Transgenic Res., 6:169-76, 1997.

Skriver and Mundy, Plant Cell, 2:503-512, 1990.

Smith, Watson, Bird, Ray, Schuch, Grierson, Mol. Gen. Genet., 224:447-481, 1990.

Southern, J. Mol. Biol., 98:503-517, 1975.

Spencer et al., Plant Molecular Biology, 18:201-210, 1992.

Sprague and Dudley, eds., Corn and Improvement, 3rd ed., 1988.

Stalker et al., Science, 242:419-422, 1988.

Stief et al, Nature 341:343 1989.

Sullivan, Christensen, Quail, Mol. Gen. Genet., 215(3):431-440, 1989.

Sutcliffe, Proc. Nat'l Acad. Sci. USA, 75:3737-3741, 1978.

Symons, Annu. Rev. Biochem., 61:641-671, 1992.

Takumi and Shimada, “Variation in transformation frequencies among six common wheat cultivars through particle bombardment of scutellar tissues,” Genes Genet. Sys., 72:63-69, 1997.

Tanksley et al., Bio/Technology, 7:257-264, 1989.

Tarczynski et al., Proc. Nat'l Acad. Sci. USA, 89:1-5, 1992.

Tarczynski et al., Science, 259:508-510, 1993.

Thillet et al., J. Biol. Chem., 263:12500-12508, 1988.

Thompson et al., “Maize transformation utilizing silicon carbide whiskers: A review,” Euphytica, 85(1-3):75-80, 1995.

Thompson et al., The EMBO Journal, 6(9):2519-2523, 1987.

Thompson, Drayton, Frame, Wang, Dunwell, Euphytica, 85(1-3):75-80, 1995.

Tian, Sequin, Charest, Plant Cell Rep., 16:267-271, 1997.

Tingay et al., The Plant Journal v. 11 (6) p. 1369-1376. 1997.

Tomes et al., “Transgenic tobacco plants and their progeny derived by microprojectile bombardment of tobacco leaves,” Plant Mol. Biol., 14:261-8, 1990

Tomic et al., Nucl. Acids Res., 12:1656, 1990.

Torbet et al., “Transformation of oat using mature embryo-derived tissue cultures,” Crop Science, 38:226-231, 1998.

Torbet et al., “Use of paromomycin as a selective agent for oat transformation,” Plant Cell Reports, 14:635-640, 1995.

Toriyama et al., Theor Appl. Genet., 73:16, 1986.

Tsukada; Kusano; Kitagawa, Plant Cell Physiol., 30(4)599-604, 1989.

Twell et al., Plant Physiol 91:1270-1274, 1989.

Uchimiya et al., Mol. Gen. Genet., 204:204, 1986.

Ugaki et al., Nucl. Acid Res., 19:371-377, 1991.

Upender, Raj, Weir, Biotechniques 18(1):29-30, 1995.

Van der Krol, Mur, Beld, Mol, Stuitje, Plant Cell, 2:291-99, 1990.

Van Eck; Blowers; Earle, Plant Cell Reports, 14(5):299-304, 1995.

Van Tunen et al., EMBO J., 7:1257, 1988.

Vasil et al., Plant Physiol., 91:1575-1579, 1989.

Vernon and Bohnert, The EMBO J., 11:2077-2085, 1992.

Vodkin et al., Cell, 34:1023, 1983.

Vogel, Dawe, Freeling, J. Cell. Biochem., (Suppl. 0) 13:Part D, 1989.

Waddington, C. H. Nature 150, 563-565 (1942).

Waddington, C. H. Evolution 7, 118-126 (1952).

Walker et al., Proc. Nat'l Acad. Sci. USA, 84:6624-6628, 1987.

Wandelt and Feix, Nucl. Acids Res., 17(6):2354, 1989.

Wang et al., Molecular and Cellular Biology, 12(8):3399-3406, 1992.

Watrud et al., In: Engineered Organisms and the Environment, 1985.

Watson and Ramstad, eds., Corn: Chemistry and Technology, 1987.

Wenzler et al., Plant Mol. Biol., 12:41-50, 1989.

Withers and King, Plant Physiol., 64:675-678, 1979.

Wolter et al., The EMBO J., 4685-4692, 1992.

Xiang and Guerra, Plant Physiol., 102:287-293, 1993.

Xu et al., Plant Physiol., 110:249-257, 1996.

Yamada et al., Plant Cell Rep., 4:85, 1986.

Yamaguchi-Shinozaki et al., Plant Cell Physiol., 33:217-224, 1992.

Yang and Russell, Proc. Nat'l Acad. Sci. USA, 87:4144-4148, 1990.

Young, J. C., Moarefi, I. & Hartl, F. U. J. Cell Biol. 154, 267-274 (2001).

Yuan and Altman, Science, 263:1269-1273, 1994.

Yuan et al., Proc. Nat'l Acad. Sci. USA, 89:8006-8010, 1992.

Zhang et al., “Agrobacterium-mediated transformation of elite indica and japonica rice cultivars,” Mol. Biotechnol., 8(3):223-231, 1997.

Zhang, McElroy, Wu, The Plant Cell, 3:1155-1165, 1991.

Zheng and Edwards, “Expression of resistance to barley stripe mosaic virus in barley and oat protoplasts,” J. Gen. Virol., 71:1865-1868, 1990.

Zhou et al., Plant Cell Reports, 12(11).612-616, 1993.

Zukowsky et al., Proc. Nat'l Acad. Sci. USA, 80:1101-1105, 1983.

We claim:

1. A method of detecting genetic variation in a plant genome, comprising inhibiting Hsp90 activity in at least one plant cell.

2. The method of claim 1, further defined as a method of identifying a plant polymorphism.

3. The method of claim 1, wherein inhibiting Hsp90 activity lowers a threshold for manifestation of a pre-existing genetic polymorphism in the plant cell.

4. The method of claim 1, wherein the at least one plant cell is in cell culture.

5. The method of claim 1, wherein the at least one plant cell is comprised in a seed.

6. The method of claim 1, wherein the at least one plant cell is comprised in a plant.

7. The method of claim 1, wherein inhibiting Hsp90 comprises administering an Hsp90-inhibiting agent to a plant cell.

8. The method of claim 7, wherein the Hsp90-inhibiting agent is administered to a plant cell that is in cell culture.

9. The method of claim 8, further comprising developing a plant from the plant cell.

10. The method of claim 7, wherein the Hsp90-inhibiting agent is administered to a plant cell that is in a seed.

11. The method of claim 10, further comprising developing a plant from the seed.

12. The method of claim 7, wherein the Hsp90-inhibiting agent is administered to a plant cell that is in a plant.

13. The method of claim 12, wherein the Hsp90-inhibiting agent is administered to the plant after the plant has sprouted from a seed.

14. The method of claim 7, wherein the Hsp90-inhibiting agent inhibits ATPase activity of Hsp90.

15. The method of claim 14, wherein the Hsp90-inhibiting agent is geldanamycin or radicicol.

16. The method of claim 7, wherein the Hsp90-inhibiting agent is photosensitive.

17. The method of claim 1, wherein the Hsp90 activity is inhibited by inhibiting expression of a nucleic acid sequence that encodes Hsp90.

18. The method of claim 17, further defined as a method of inhibiting transcription of a nucleic acid sequence that encodes Hsp90.

19. The method of claim 17, further defined as a method of inhibiting translation of Hsp90.

20. The method of claim 1, wherein Hsp90 activity is inhibited by increasing Hsp90 degradation.

21. The method of claim 1, wherein Hsp90 activity is inhibited by decreasing Hsp90 half-life.

22. The method of claim 1, wherein the Hsp90 activity is inhibited by knocking out a gene encoding Hsp90 in the cell's genome.

23. The method of claim 1, wherein genetic variation is identified by examining the phenotype of the plant cell.

24. The method of claim 23, wherein examining the phenotype of the plant cell comprises examining the morphology or physiology of the cell.

25. The method of claim 23, wherein examining the phenotype of the plant cell comprises examining the morphology or physiology of a plant comprising the cell.

26. The method of claim 25, wherein examining the phenotype of the plant cell comprises examining the morphology of a plant comprising the cell.

27. The method of claim 23, wherein examining the phenotype comprises examining cotyledon morphology, adult leaf morphology, hypocotyl morphology, root morphology, root hair morphology, and/or rosette morphology.

28. The method of claim 25, wherein examining the phenotype of the plant cell comprises examining the physiology of a plant comprising the cell.

29. The method of claim 28, wherein examining the phenotype comprises examining anthocyanin accumulation and/or increased response to gravity.

30. The method of claim 23, wherein examining the phenotype comprises exposing a plant comprising the cell to a stimulus and assaying the plant for a reaction to the stimulus.

31. The method of claim 30 wherein the stimulus is temperature, light, gravity, salinity, metal, touch, sound, humidity, nutrient concentration, growing conditions, dark, and/or a change in any of these.

32. The method of claim 1, wherein genetic variation is detected by examining the genotype of the cell.

33. The method of claim 1, wherein the plant cell is a cell of a monocot.

34. The method of claim 1, wherein the plant cell is a cell of a dicot.

35. The method of claim 1, wherein the plant cell is a cell of cotton, rice, barley, oats, canola, soybean, corn, wheat, rye, tobacco, sorghum, Arabidopsis thaliana, sunflower, alfalfa, tomato, potato, sugar beet, cassaya, broccoli, cauliflower, spinach, peanut, olive tree, grass, rose, carnations, daisies, orchids, tulips, irises, palms, ferns, ficus, evergreen, ivy, grapes, hops, aloe vera, opium poppy, sweet potatoes, yams, Echinacea, witch hazel, or Gingko biloba.

36. The method of claim 34, wherein the plant cell is a cell of Arabidopsis thaliana.

37. The method of claim 1, wherein the plant cell is a cell of a crop plant.

38. The method of claim 37, wherein the crop plant is cotton, corn, sorghum, soybean, tobacco, rice, canola, wheat, rye, spinach, grapes, peanut, or mustard.

39. The method of claim 1, further defined as a method of detecting genetic variation among a plurality of plant cells.

40. The method of claim 39, further defined as a method of detecting genetic variation among a plurality of plant cells comprised in a single plant.

41. The method of claim 39, further defined as a method of detecting genetic variation among a plurality of plant cells comprised in a plurality of plants.

42. A plant manifesting a pre-existing genetic polymorphism and having inhibited Hsp90 activity.

43. The plant of claim 42, wherein said inhibited Hsp90 activity is a result of application of a pharmacological Hsp90 inhibitor.

44. The plant of claim 42, wherein said inhibited Hsp 90 activity is a result of a transgenic event.

45. The plant of claim 42, wherein the manifestation of said polymorphism results in a phenotype related to cotyledon morphology, adult leaf morphology, hypocotyl morphology, root morphology, root hair morphology, and/or rosette morphology.

46. The plant of claim 42, wherein said plant is cotton, rice, barley, oats, canola, soybean, corn, wheat, rye, tobacco, sorghum, Arabidopsis thaliana, sunflower, alfalfa, tomato, potato, sugar beet, cassaya, broccoli, cauliflower, spinach, peanut, olive tree, grass, rose, carnations, daisies, orchids, tulips, irises, palms, ferns, ficus, evergreen, ivy, grapes, hops, aloe vera, opium poppy, sweet potatoes, yams, Echinacea, witch hazel, or Gingko biloba.

47. A plant having a manifested genetic variation, wherein said genetic variation has been manifested via inhibition of Hsp90 activity.

48. A method of identifying genetic variation in the genome of a plant, comprising inhibiting Hsp90 function in a plant cell to an extent sufficient to reveal genetic variation not detectable in the plant cell in the absence of inhibition of Hsp90 function; maintaining the plant cell under conditions appropriate for growth of the cell and production of a plant and determining whether the resulting plant exhibits a phenotype different from a control plant produced from the same cell type, in which Hsp90 function is not inhibited, wherein if the resulting plant exhibits a different phenotype from the phenotype of the control plant, genetic variation has been identified.

49. The method of claim 48, wherein the plant cell is a plant seed cell.

50. The method of claim 49, wherein the plant seed cell is an F1 seed cell.

51. The method of claim 50, wherein the plant cell is a cell from a plant selected from the group consisting of: corn, cotton, rice, barley, oats, canola, soybean, wheat, rye, tobacco, sorghum, Arabidopsis thaliana, sunflower, alfalfa. tomato, potato, sugar beef cassaya, broccoli, cauliflower, peanut, olive tree, grass, rose, carnation, daisy, orchid, tulip, iris, palm, fern, focus, evergreen, ivy, grape, hops, aloe vera, opium, poppy, sweet potato, yam, Echinacea, witch hazel and gingko biloba.

52. A method of identifying genetic variation in a plant, comprising growing a seed under conditions that inhibit Hsp90 function in the seed to an extent sufficient to reveal genetic variation in the seed not detectable in the absence of inhibition of Hsp90 function, thereby producing a plant and determining whether the plant exhibits a phenotype different from the phenotype of a plant grown from the same type of seed in the absence of inhibition of Hsp90, wherein if the phenotypes of the two plants differ from one another, genetic variation has been identified.

53. The method of claim 52, wherein the seed is a seed from a plant selected from the group consisting of: corn, cotton, rice, barley, oats, canola, soybean, wheat, rye, tobacco, sorghum, Arabidopsis thaliana, sunflower, alfalfa. tomato, potato, sugar beef cassaya, broccoli, cauliflower, peanut, olive tree, grass, rose, carnation, daisy, orchid, tulip, iris, palm, fern, ficus, evergreen, ivy, grape, hops, aloe vera, opium, poppy, sweet potato, yam, echinacea, witch hazel and gingko biloba.

54. The method of claim 53, wherein the conditions that inhibit Hsp90 function are selected from the group consisting of: growth in the presence of a chemical agent that inhibits Hsp90 function and growth in the presence of an environmental change.

55. The method of claim 54, wherein the chemical agent is a drug and the environmental change is a moderate temperature increase or an increase in density of substratum on which the seed is grown.

56. The method of claim 55, wherein the drug is geldanamycin or radicicol.

57. A method of revealing genetic variation in a plant, wherein the genetic variation is not revealed in the plant in the presence of normal Hsp90 function, comprising growing seeds from which the plant grows under conditions that cause inhibition of Hsp90 function in the seeds sufficient to produce plants that exhibit a phenotype not exhibited by plants grown from the seeds in the absence of the conditions that cause inhibition of Hsp90 function, whereby genetic variation in the plant is revealed.

58. The method of claim 57, wherein the conditions that cause inhibition of Hsp90 function comprise growing the seeds in the presence of a drug that inhibits Hsp90 function.

59. The method of claim 58, wherein the drug is selected from the group consisting of geldanamycin and radicicol.

60. The method of claim 59, wherein the conditions that cause inhibition of Hsp90 function comprise growing the seeds under a temperature sufficiently elevated to inhibit Hsp90 function.


SEQUENCE LISTING (8 sequences)

SEQ ID NO: 1 - DNA, 3413 bp, Arabidopsis thaliana
ctagtatcatagtgagagattagctattgctttgactgatcacgttccagctgatagaccgatccgagtaatgaagaatctgctgagtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgttgctcacaaagctaaaaacggttgatttcatcgaaatagggcttcgttttcaaagaagaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgttttcgttgaatcaaagttcgttgcgatggcggatgttcagatggctgatgcagaaacttttgctttccaagctgagattaaccagcttcttagcttgatcatcaacacgttctacagcaacaaagaaatcttcctccgtgagctcatcagtaactcttctgatgtaagtttcccttcaaatctctctctgactcggtgtgactcgtccgcttcctattttcttgactgttgtttgttctttaattcctggattcgttgatagcgttggattcgtaggtttagcgttgtgattgcttattcaaataaatcgtgatttggcttgtgcatcacgttaagtttagaattcttagcttgtgctcgatcttcatgtgttgtagttacatatatagaacggttcttgcttcgatgtagtttttgatttaccctagaggattgagtaaagcttctgattatctttgtttatatgaacggttttgtaggctcttgacaagattcgatttgagagcttaacggataagagcaagctcgatggacagcctgaactcttcattagattggttcctgacaagcctaataagacgctctcaattattgacagtggtattggcatgaccaaagcaggtaacgaatcaatgcctaataatctctcgttggtgagatgtttagtgtatgtgctgtggttatgactctctattatttttcagatttggtgaacaacttgggaaccattgcgaggtctggaacaaaagagtttatggaggcgcttcaagctggagctgatgtaagcatgataggacaatttggtgttggtttctactctgcttatcttgttgcagagaaggttgttgtcactacaaagcacaatgatgatgaacaatacgtttgggagtctcaagctggtggttccttcactgtcactagggatgtggatggggaaccacttggtagaggaactaagatcagcctcttccttaaggacgatcaggtaaggaatcgtagctttgagtgttttgggggatgttcttttcttttggtgttttctgtgttcttacaagtgtgtttattcatgcagcttgaatacttggaggagaggagactcaaagacttggtgaagaagcactctgagttcatcagttaccctatctacctttggaccgagaaaaccaccgagaaggagatcagtgacgatgaggatgaagatgaaccaaagaaagaaaacgaaggtgaggttgaagaagttgatgagaagaaggagaaagatggtaaaaagaagaagaaaatcaaggaagtctctcacgagtgggaactcatcaacaagcagaaaccgatctggttgaggaagccagaagagatcactaaggaagagtatgctgctttctacaagagcttgaccaatgactgggaagatcacttagccgtgaaacacttctcagtggagggtcagctagaattcaaggccattctctttgtaccaaagagagctccgtttgatctctttgacacgaggaagaagttgaataacatcaagctttatgtcaggagggtgttcattatggacaactgtgaagagctaatcccagagtacctcagctttgtgaaaggtgttgttgactctgatgacttgccactcaacatctctcgtgagacgcttcaacagaacaagatccttaaggtgatcaggaagaatctagtgaagaagtgcattgagatgttcaacgagattgctgagaacaaagaggactacaccaaattctatgaggctttctccaagaatctcaaattgggtatccatgaagacagtcagaacaggggaaagattgctgatcttctacggtaccactccacaaagagtggtgatgaaatgacgagcttcaaagattacgtcacaaggatgaaggaaggtcaaaaggacattttctacatcactggtgaaagcaaaaaggcggtggagaattccttcttggagaggctgaagaagagaggctacgaggtactttacatggtggatgcgattgacgaatacgctgttggacaattgaaggagtatgacggtaagaaacttgtttctgcgactaaagaaggcctcaaacttgaagatgagaccgaagaagagaagaaaaagagggaagagaagaagaagtccttcgagaatctctgcaagacgattaaggaaattctcggggacaaggttgagaaggttgtggtctcagacaggattgtggactctccctgctgtctagtaactggtgaatatggatggactgcaaatatggagaggattatgaaggcacaggccttgagagatagcagcatgagtggttacatgtcgagcaagaaaacaatggagatcaaccccgacaacggtataatggaggacctcaggaagagagctgaagcagacaagaatgacaagtctgttaaagatcttgtcatgttgctgtatgagacagctttgttgacgtctggatttagtcttgatgaaccgaacacttttgctgctaggattcacaggatgttgaagttgggtctgagtattgatgaggatgagaacgttgaggaagatggtgatatgcctgagttggaggaggacgctgctgaagagagcaagatggaggaagtcgactaagagatgaagaaattgctcttatggttctgaaaacttctaatatgtcgagttgttctgagttttaagattttccaaaatgtctgtctttttttttttatctttagagttacttgaacattgtgactacttctagggttgggtttgtgtcaggtctgttatatcgtgtggtgggtctgtctaatactgattcgagttttg

SEQ ID NO: 2 - protein, 704 aa, Arabidopsis thaliana
MADVQMADAETFAFQAEINQLLSLIINTFYSNKEIFLRELISNSSDALDKIRFESLTDKSKLDGQPELFIRLVPDKPNKTLSIIDSGIGMTKADLVNNLGTIARSGTKEFMEALQAGADVSMIGQFGVGFYSAYLVAEKVVVTTKHNDDEQYVWESQAGGSFTVTRDVDGEPLGRGTKISLFLKDDQLEYLEERRLKDLVKKHSEFISYPIYLWTEKTTEKEISDDEDEDEPKKENEGEVEEVDEKKEKDGKKKKKIKEVSHEWELINKQKPIWLRKPEEITKEEYAAFYKSLTNDWEDHLAVKHFSVEGQLEFKAILFVPKRAPFDLFDTRKKLNNIKLYVRRVFIMDNCEELIPEYLSFVKGVVDSDDLPLNISRETLQQNKILKVIRKNLVKKCIEMFNEIAENKEDYTKFYEAFSKNLKLGIHEDSQNRGKIADLLRYHSTKSGDEMTSFKDYVTRMKEGQKDIFYITGESKKAVENSFLERLKKRGYEVLYMVDAIDEYAVGQLKEYDGKKLVSATKEGLKLEDETEEEKKKREEKKKSFENLCKTIKEILGDKVEKVVVSDRIVDSPCCLVTGEYGWTANMERIMKAQALRDSSMSGYMSSKKTMEINPDNGIMEDLRKRAEADKNDKSVKDLVMLLYETALLTSGFSLDEPNTFAARIHRMLKLGLSIDEDENVEEDGDMPELEEDAAEESKMEEVD

SEQ ID NO: 3 - DNA, 2397 bp, Wheat
cgcggccgcgtcgacgaacccgagaatcccccgctccaatagccatctcctctcgccccagccctcgtcgagcgcgcgcgttccaggagcttccggtgccggcggcgccatggcttcggagaccgagaccttcgccttccaggccgagatcaaccagctgctctcgctcatcatcaacacctcctactccaacaaggagatcttcctccgtgagctcatctccaacgcctccgatgcgttggataaaatcaggtttgagagcctgactgacaagagcaagctggatgctcagccagagctgttcatccgtataatccctgacaaggccacgaacacactcacacttattgacagcggtattggtatgaccaagtcagacctcgtgaacaaccttgctaccattgggaggtctggcaccaaggatttcatggaggcactggctgctggtgctgatgtatccatgattggccagtttggtgtcggtttctactctgcttacctcgttgctgagagagtcattgtgaccagcaagcacaacgatgacgagcagcatgtgtgggagtcccaggctggtggttccttcactgtgacacgtgatactactggagagccccttgggaggggtactaagatcaccctctacctcaaggatgatcagttggagtaccttgaggagcgtcgccttaaggatctggtgaagaagcactctgagttcatcagctatcccatctctctctggacggagaagaccactgagaaggaaatttctgacgatgaagatgaggatgagaagaaggatactgaggagggcaagtttgaggaaattgatgaagagaaggaagaaaaggagaagaaaaagaagaagatcaaggaggtttctcacgagtggaacctgatcaacaagcagaagcccatctggatgaggaagcctgaggagatcacaaaggatgagtttgctgccttcttcaagagcctgacaaacgactgggaggagcaccttggcgtcaagcacttctctgtggagggtcagcttgagttcaaggccgtcctcttcgttcccaagagggccccctttgacctttttgacaccaggaagaagctcaacaacatcaagctctatgtgcgccgtgtcttcatcatggacaactgtgaggagctgatcccagagtggctgagctttgtcaagggcattgttgactctgaggatcttcccctgaacatctctcgtgagacactccagcagaacaagatcctcaaggtcatccggaagaaccttgtgaagaagtgcatcgagctcttctttgagattgccgagaacaaggaggactacaacaagttctatgaggctttctccaagaacctcaagcttggcgtccacgaggactccaccaacaggaccaagcttgctgagctcctgaggtaccactccaccaagagcggtgatgagcttaccagcctcaaggactacgtgacgaggatgaaggagggccagaacgacatctactacatcaccggtgagagcaagaaggctgtggagaattctcccttccttgagaagctgaagaagaagggctatgaggtgctgtacatggtggacgccattgatgagtactccattggccagctcaaggagtttgagggaaagaagctggtctctgccactaaggagggcctgaagcttgatgacagcgaggaagagaagaagaggaaggaggagctcaaggagaagtttgaggggctctgcaaggtgatcaaggaggtgctgggcgacagggtcgagaaggtgattgtctctgaccgcgtcgtcgactcgccgtgctgcctggtgaccggcgagtatggctggactgccaacatggagaggatcatgaaggcccaggccctgagggacacgagcatgggcggctacatgtcgagcaagaagacgatggagatcaacccggagaacgccatcatggaggagttgcgcaagcgcgccgacgctgacaagaacgacaagtctgtcaaggacctggtgatgctgctgttcgagaactccctgctcacctctggattcagcctcgacgaccccaacaccttcggcaccaggatccaccgcatgctcaagctgggcctgagcatcgacgaggacgaggaggctgctgaggctgacacggacatgccgccgctggaggaggacgccggcgagagcaagatggaggaggtcgactaggcgtgtgcgttcgcttgctatccgagtcgtagagctagcttatcttttgggttactgtcgcgtggtcggtttgtcttttatggtttactgtgcctagcctgctgtaatggggctaggggagtggtctgtggagtccccggaactgcgtatcgtaatcgttgcttatatatatcgtctttactacc

SEQ ID NO: 4 - DNA, 1610 bp, Tomato
ataccttgcagtcaaacacttctctgttgaggggcaacttgaattcaaggcaatcctctttgtacctaagagggctccatttgatctatttgacacccgcaagaagatgaacaacatcaaactttatgtcaggagggtgttcatcatggacaactgtgaggaacttatccctgagtaccttggattcgtgaagggtgttgttgactctgatgatttgcccctcaatatctcccgtgaaatgctgcagcagaacaagattctcaaggtcattaggaagaacctcgtgaagaaatgtattgagatgttcaatgagattgcagagaacaaggaggactacaacaagttctacgaggctttctcaaagaacttgaagctgggcattcatgaagatagccagaacagggctaagttggctgacttccttcgatatcagtcaacccaagagtgtgatgagctgacaagtttgaaagattatgtaaccaggatgaaggaggttcagaaagacatctactacatcactggagagagcaaaaaggcagttgaaaattcaccattcttggaacgcctaaagaagaaaggatatgaagtactcttcatggttgatgccattgatgaatatgctattgggcaactgaaggaatatgatggtaagaaactggtttctgttacaaaggagggactgaagctcgatgacgagagcgaagaagaaaagaagaaaaaggaagagaaaaaacaatcctttgagagcctttgcaaggtcatcaaggacattcttggagacaaagttgagaaggttgtagtctctgataggattgttgattctccatgttgcttagtgacaggtgagtatggttggacagctaacatggaaaggatcatgaaagctcaagctttgaaggacaatagcatgagctcttacatgtctagcgagaagacaatggaaatcaaccctgataatggcattgtggaggagttgaggaagagagctgaagttgacaagaatgacaagtcggtgaaagatcttgtgctgctgctgtttgagacagctttgctaacatctggttttagtcttgatgacccgaatacatttgctgcaagaattcatagaatgctgaagttgggtttgagcattgacgaagaagaggaagctggtgtggatgttgatgatatgcctcctctggaggatgttggtgaggaaagcaagatggaagaagtggactaatcattgaaatcagttgagacttttgagatgcgtgaaaatacaatttgagcgtgttgctttttttctcatcttgtgtctagtatagtttttttttttaggcaagaaaagctgttcaatcaaaatgatcaattaacaaaggttggcatattacattgaaagtaactttagtttcatgggtgtcaactgggactttggaccaaagccttatgcaactaacccttcacaacaacctgaagtactcacaattacaattacaacatagtgttgatagacaaaaggtaaagaagtgataaaacaattatccaactgtatattggagaattgcttagttgctgccaccgccgaagagataacccaaagaagatccacctccaggatttgccatagg

SEQ ID NO: 5 - DNA, 2731 bp, Rice
gggggagtcaagccgcggcgaggcgacgaactcatcacctccactctctgctgcaacacgagtgaatcggaggcggcacgatgcgcaagtgggcgctctcctccgcgctcctcctcctcctcctcaccacactccccgatccagctaagaagctccaagtcaatgctgatgacagcactgatgagttagttgatccgccgaaggtagaggagaagattggtggtgttccccatggcctgtccacggactctgaggttgttcagagggaggctgagtcaatctcgaggaagaccctcaggagctcagcggagaaatttgagttccaggctgaggtgtccagactcatggacatcatcatcaactcactctacagcaacaaggacattttcctgagggagctcatctccaatgcttctgatgctttggacaagattaggttcctggctctcactgataaggaggttttgggcgaaggtgacactgctaagcttgaaatccagattaagctggataaggagaagaagattctctccattcgggataggggtattggtatgaccaaggaagatctcattaagaaccttgggaccattgccaaatctggaacttcagcttttgtggaaaaggtgcagactgggggtgacctcaatctcattgggcaattcggtgttggtttctattcagtatacttggttgctgactatgttgaagtgatcagcaagcacaatgacgacaaacagcatgtctgggagtccaaagctgatggatcattcgctatctctgaggatacatggaatgaacccctgggccgtggaactgaaataagactgcacctccgagatgaagccaaggagtacgtggaagaagacaagctaaaggatttggtgaagaagtactctgagttcatcaatttccccatttacttgtgggcaaccaaggaggttgatgtggaagtgccagctgatgaggatgagtcaagtgaatcaagtgaagaagaggaatcatcccctgagtctacagaggaagaagagacagaagagggtgaagagaagaagcccaagacaaagacagtaaaggaaaccactactgagtgggagcttctgaatgatgtgaaggctatatggcttcgtagccctaaggaggttactgaagaagaatacacaaagttttaccactcacttgctaaggactttggtgatgacaagcccttgtcttggagccacttcactgctgagggagatgttgagttcaaagctttgctctttgttcctcccaaggctccacatgatctctatgagagttactacaactctaacaagtcaaaccttaagttgtatgttagaagggtttccatctctgatgaatttgatgagcttcttccgaagtatctcagctttttgaagggtctcgtcgactcggacacattgcctctcaatgtatcacgagaaatgctccaacaacatagtagcctgaagaccatcaagaagaaactaatccgcaaagctcttgacatgattagaaaacttgctgaggaagatcctgatgagtacagcaacaaagataagacagatgaagaaaaaagtgcaatggaggagaaaaagggccagtatgccaagttctggaatgagtttggaaaatcagtcaagttgggcatcattgaagacgcaactaacaggaaccgtcttgcaaagcttctgagatttgaaagtaccaagtcggaaggcaaacttgcctctcttgatgagtacatttcaaggatgaagccagggcagaaggacatcttttacattaccggaagcagcaaggaacaattagagaaatcaccgttccttgagaggctaaccaaaaagaactacgaggttatctacttcactgaccctgttgacgagtacttgatgcaatacctcatggactacgaggacaagaagttccagaacgtctccaaggagggtctcaaactcggcaaggactctaagctcaaggacctcaaggagtccttcaaggagctgaccgactggtggaagaaggctcttgacaccgagagcgtggactcggtgaagatcagcaaccggctgagcgacaccccctgcgtggtggtgacatccaagtacgggtggagcgccaacatggagaagatcatgcagtcacagaccctctcggatgccagcaagcaggcgtacatgcgcggcaagagggtactcgagatcaaccccaggcaccccatcatcaaggaactccgtgacaaggtcgcccaggacagcgagagcgagagcttgaagcagacggcgaagctggtgtaccagacggccctcatggagagcggcttcaacctccctgatcccaaggacttcgccttcagcatctacaggtcggtgcagaagagccttgacctgagccccgacgcggccgtggaagaggaagaggaggtcgaggaggccgaggttgaagagaaggaatcatccaacatcaaggaggaggcggagccgtcgtcgtatgataaggacgagctgtagtgatgtgtcgccaccaactgttgtttttttatctagctcttgtgttaggttgtttaaaaatgtgatggtttcgagggacgtccatttttccatgcgaaaatgcatagtaccatgatacggggattttgtcgatgtacgtagtttgtgatactctagttcaaattaatggccgtctaatttcgatcaagaaaaaaaaaaaaaaaaaagaaaaaaaaaaa

SEQ ID NO: 6 - DNA, 2670 bp, Catharanthus roseus
agtcgatgaggaagtggacggtaccttccgttctgtttctattgtgcccttctctttcttcttcctgtcaaggtaggaagatccatgcaaatgcagaagctgattctgatgctccagtagatccgccaaaggtggaagataagattggagctgttccaaatggtttatctaccgattcagatgttgcgaaaagagaagcagaatcaatgtcaatgagaaatctccgttctgatgcggagaagtttgaattccaggctgaggtttctcgacttatggatatcattatcaattctctttacagtaacaaggacatctttttgagggagttgatatctaatgcttcggatgcattagacaagattagattcctcgccctcactgataaagaaatattgggtgaaggtgacactgctaaacttgagattcagattaagctagacaaagaaaagaaaatactttctattcgtgacagaggtataggtatgacaaaagaagatttaatcaaaaatttgggaaccatagcaaagtctggaacttcagcttttgtggagaaaatgcagactagtggggatctcaacctcattgggcaatttggtgttggtttttactctgtataccttgttcctgactatgttgaagtcattagcaaacacaatgatgacaaacagtatatttgggagtctaaggctgacggggcatttgcaatttctgaggatgtctggaatgaaccccttggccgtggaacagaaataaggttgcatcttagagatgaggcacaggaatacttggatgagttcaagctgaaggaactagtgaaaaggtattccgaattcatcaattttccaatttatctctgggcaagcaaggaagtggaggtagaggttcctgctgaggaagacgattcaagcgatgatgaggataacaaatctgaaagcagctcctcagaggagggcgaagaagaagaaaccgagaaagaggaagatgagaaaaagcccaagacaaagaaggtgaaggagacaacttatgaatgggagcttctgaatgatatgaaggccatttggttaaggaatccaaaggatgtcacagatgatgagtacaccaaattttatcactctctagccaaggatttcagtgaagagaaaccactagcatggagtcacttcactgctgaaggtgatgtcgagttcaaggcttttactcttttgcctcctaaggcaccacaagatttgtacgagagctactataactcaaacaagtccaacttgaagttgtatgtcagacgtgtcttcatctctgatgaatttgatgaacttttgccaaaatatctcaactttttgaagggtcttgttgattctgataccttgccacttaatgtatcgagagaaatgctccagcaacatagcagcttaaaaactataaagaagaaactcataagaaaggcacttgatatgatccgcaagattgcagatgaggaccctgatgaagctaatgacaaggataagaaagaggttgaagaatcaactgacaatgatgagaagaagggtcagtatgcgaaattctggaatgaatttggcaagtcaattaaactgggtatcattgaggatgctgctaatagaaaccgcttggcaaaacttctccggtttgagagtacaaaatcagaggggaagcttacttcactagatcaatatatctcaagaatgaagtcaggacagaaagatatcttttacattaccggaacaagcaaagaacaattggagaaatctcctttcctcgagaggcttacaaagaaaaattatgaggttatcctcttcacagacccagtcgatgagtacctgatgcaatatctaatggactatgaggataagaaattccaaaatgtgtctaaggagggcctgaaaatcggaaaggattcgaaagacaaggaactcaaggagtcattcaaggagttaaccaagtggtggaagggtgcccttgctagcgaaaatgtagatgacgttaagataagcaaccgtttggcaaacacaccctgcgttgttgtgacatcaaagtacggttggagttcaaatatggaaaggatcatgcagtcacaaaccttatcagatgctagtaagcaggcatacatgcgaggaaagagggtactggaaatcaatccccgacacccaatcattaaagagcttcgagagagagttgtgaaggatgctgaggatgaaagcgtaaaacaaacagcacggctcatgtaccagaccgcactgatggagagtggcttcatgctcaatgatcccaaagaattcgcctccagcatttacgactctgtaaaaagtagtctgaaaatcagccccgatgctactgttgaagaggaagatgatactgaggaagctgaggctgaatccggcacaacagaatcatcggcagctgaagatgctggtgccgagactttggatctcaaggacgaattgtagaattttttggcagaagtttctgaaagaggatctcattttagcttcctctcagctgagaaaactgtccctctctctagcgtaatttgtattagtagctctccattgaggaactagttcacgggtgattcaagttttaggtgcttgtaatcttaaagcgtcaggaattttgattttgtttctttttttacttttccagaaagtgatagtcaag

SEQ ID NO: 7 - DNA, 2654 bp, Rye
tcccaaacaccgcgccggaaccccaaaccatccgaacaaccaaccaccagcgagcgcggtaggctcggcgctgttggcccagcaccgtccgtctctctctcatggcgcccgcgctgagcaggaccctgggcccgtcctcggtggcggcgctgcggccgagcccgtcgcgggggctgccgctggccgcgctacttccacagggtaagaggtcttccagcgccagaggagtcaggtgggaggccgggaggggcaggctggtcggggcgaggtgcgcctccgccgtcgccgagaagaccgccggcgaggaggaggaggcggccggcgagaagttcgagtaccaggccgaggttagccggttgatggatttgattgttcacagcctgtacagccacaaggaagtgttccttcgggagcttgtaagtaatgcgagtgatgcgctggataagctgagatttctcagtgtaactgactcatccgtgctggctgatggtggtgagctggaaataaggattaaacctgaccctgatgctggaacaataacaatcactgatagtggcattgggatgacaaaggatgagctcaaggactgccttggaactattgcgcaaagtggtacctccaaatttttgaaggctctgaaggagaacaaggagcttggtgcagataatggacttatcggtcaatttggtgttggattttattcggcttttcttgttgcagagaaggttgtggtgtccactaagagtcccaagacagataagcagtatatatgggaagctgaggctaacagtagttcatacgttatcagggaagagactgatcctgagaaaatgctgacacgtggaacacagattaccctctttttaagagaggatgataagtatgaatttgctgaccctgcgaggattcaaggtctagttaagaactactcgcagtttgtttcattccccatctttacatggcaagagaaatctagaacagttgaggttgaagaagaggagtcaaaagaaggcgaagagacagcagagggcgagaaggaggagaagaagaaaactatcactgagaagtactgggattgggagttggcaaatgaaacaaagccaatttggatgagaaatccaaaggaagttgaggaaacagagtacaatgaattttacaagaaggcattcaatgagtttttggatcctctagcccatgcacacttcacaacagagggtgaggtggaatttaggagtgttctctacatccctggaatggcaccacttagcaatgaggagataatgaaccctaagaccaaaaatatccgattgtatgtcaagagggttttcatttctgatgactttgacggcgagctgttccccaggtacttgagctttgtcaaaggtgttgttgactcgaatgatctccctctgaatgtttctcgtgagattcttcaagaaagtcgcattgtcaggatcatgcgcaagagacttgttaggaagacttttgatatgattcaggatattgctgacaaggataacaaggaggactacaagaaatttggggagagctttggcaaatttatgaaacttggttgcattgaggactcaggaaatcagaagcgccttgctcctttgctgcggttttattcctccaaaaatgaaacggatttgataagtctcgatcagtatgtggagaatatgcccgagacccaaaaggcaatctattatattgccacagatagtcttcagagtgcaaagaccgctccattcttggaaaagctgcttcagaaagatattgaagttctctaccttattgagccgattgatgaggtagccattcagaatctgcagacatacaaagagaaaaagtttgttgatatcagcaaggaagacctagaattgggcgatgaggatgaggacaaggaagaaaccaagcaggaatacactcttctttgtgactggataaagcagcagctgggtgacaaagttgccaaggtccaaatttcgaagcgactcagctcttcaccatgtgttcttgtatctggcaagtttggttggtcggctaacatggaaaggcttatgaaagcacaaacgcttggtgacacatcaagcttggagttcatgagaggaaggagaatttttgaaatcaaccccgaccaccctattgtcaaggacttgagtgctgcttgcaagaatgagcctgatagcaccgaagctaagagggcagttgagctgctgtacgagactgccctgatctccagcggttatactcctgagagtccagcggagttggggggcaagatctacgagatgatgaccatcgcccttggcgggagatggggtagatccggcgccgaggaggctgagaccaatgtcgacggtgactcttctgaaggtgttgtaccggaggtcattgagccatccgaagtcaggactgagaacgagaatgacccatggagagattagttagttctgtaggcatccattatttttttaaataccctggaggtggacgagcaaaaaataggagtatcactggagccagagtgatgatgacatcgagcgatggactgtctttgtaagaacactgagctccagaatcttgtttgttatcgcccaaggataaattttgtaggtgacttttttgacggccgtgaaattaaattgtctttg

SEQ ID NO: 8 - DNA, 2682 bp, Barley
actctgcctgctgcaaccaaaactggtgagtgaagcggccggcgacgatgcgcaagtgggcgctctcctgcgcgctcctcctcgtcctcctcctcaccaccctccccgatccagctaagaagctccaagtcaatgccgaggagagcagcgacgaggttggcgattttcccaaggtagaggagaagcttggggccgtgccccatggcttgtccaccgactccgaggttgtccagagggagtccgagtcgatctcgaggaagaccctcaggaactcggccgagaagtttgagttccaggccgaggtgtccaggctcatggacatcatcatcaactcactctacagcaacaaggacatcttcctgagggagctcatttccaatgcatctgatgctttggacaagattaggttccttgccctcaccgataaggaggttatgggtgaaggtgacactgctaagcttgaaatccagattaagttggataaggagaacaagattctctctattcgcgataggggtgttggtatgaccaaggaagatctcattaagaaccttggaaccattgccaagtctggaacttcagcttttgtggaaaaaatgcagactggaggtgacctcaatctcattgggcagtttggtgttggtttctactcagtatacctggttgctgactatgttgaagtggtcagcaagcacaacgatgacaaacagtatgtatgggagtccaaagctgatggatcattcgctatttctgaggatacatggaatgaacccctgggccgtggaactgagatcaaactgcatctccgcgatgaggctaaggagtacttggaagaaggaaagctgaaggacttggtgaagaagtactctgagttcatcaacttccccatctacttgtgggcaaccaaggaggttgatgttgaagtgccagctgatgaggaggaatcaaacgaagaggaggaatcaaccacagagaccacagaggaagaagagacagaagatgatgaagagaagaagcctaagacaaagactgtaaaggaaactactactgaatgggagcttcttaatgatatgaaggccgtgtggcttcgtagccccaaggaggttaccgaagaggagtatgcaaagttttaccactcacttgctaaggactttggtgacgacaagcctatgtcttggagccacttcagtgctgagggagatgttgagttcaaagctttgctctttgttcccccgaaggctccacatgatctgtacgagagctactacaatgctaacaagtcaaacctcaagctgtatgttagaagggttttcatctctgatgaattcgatgatcttcttccaaaatacctcagctttttgatgggtattgttgactcagacacattgcccctcaatgtatcccgagaaatgcttcaacaacatagcagtctgaagaccatcaagaagaaactgatccgcaaggctcttgacatgattaggaaacttgctgaggaggatcctgatgagtacagcaacaaagaaaagacagatgatgaaaagagtgcaatggaggagaagaagggccagtatgccaagttctggaacgagtttggcaaatcagtcaagctgggaatcattgaagatgcaacaaacaggaaccgtcttgcaaagcttctgagattcgagagttccaagtcagatggcaaacttgtctcccttgatgagtatatttcaaggatgaaatcagggcagaaggacatcttctaccttacaggaagcagcaaggaacagctagagaaatctccattccttgagcagctaaccaagaaaaactacgaggttatctacttcaccgaccccgtggatgagtacctgatgcaatacctcatggactacgaggacaagaagttccagaacgtgtccaaggagggcctgaagctcggcaaggactcgaagctcaaggacctcaaggagtcgttcaaggagctgaccgactggtggaagaaggccctggacacggagggcattgactctgtgaagatcagcaaccggctgcacaacaccccctgtgtggtggtcacctccaagtatggatggagctccaacatggagaagatcatgcaggcacagaccctgtcggacgcgagcaagcaggcgtacatgcgcggcaagagggtcctggagatcaacccccggcaccccatcatcaaggagctccgcgacaaggtcgcccaggacagcgacagcgagggcctgaagcagacggcgaggctggtgtaccagacggcgctgatggagagcgggttcaacctgccggaccccaaggactttgcgtcgagcatctaccggtcggtgcagaagagcctggacctgagccccgacgctgccgtggaggaggaagaggaggtggaggagccagaggtggaggagaaggagtcggccaagcaggaggcggaggagccggaacacgagcagtacgacaaggacgagctgtagttgctcatatcgcccatttagctttcatttcattagaaagaaatgttggactggaaatgtgatggttttgagggacgtccatttttaccgcgcggaagacgattaatcatcgatcgtgatacggggggttttgtccgtgtaatgtactactgtacctagtttcttgtttaatgacgataccctagctagttggaattagcgtgca